(19) United States
(12) Patent Application Publication
(10) Pub. No.: US 2026/0295110 A1
(43) Pub. Date: Oct. 1, 2026

(54) WOUND DRESSING

(71) Applicant: T.J.Smith and Nephew,Limited, Hull (GB)

(72) Inventor: Anthony Colin Dagger, York (GB)

(21) Appl. No.: 19/474,977

(22) PCT Filed: Apr. 11, 2024
(86) PCT No.: PCT/EP2024/059793

§ 371 Date: Oct. 13, 2025

(30) Foreign Application Priority Data
Apr. 12, 2023 (GB) 2305320.0

(51) Int. Cl. A61L 15/44; A61F 13/00; A61L 15/20; A61L 15/24

(52) U.S. Cl. CPC A61L 15/44; A61F 13/00063; A61L 15/20; A61L 15/24; A61F 2013/00225; A61L 2300/114; A61L 2300/404; A61L 2300/412

(57) ABSTRACT

Disclosed embodiments relate to a wound dressing which can generate nitric oxide. The wound dressing may include a cover layer, an activator layer such as an acid providing layer, and a nitrite providing layer. The activator layer may include acidic groups and may be hydrogel, xerogel, or other suitable material. The nitrite providing layer may include a mesh component embedded with a nitrite salt and an antioxidant/reducing agent such as sodium iso-ascorbate. Nitrite ions of the nitrite providing layer may react with the acidic groups of the activating layer to generate nitric oxide. Additionally, the wound dressing may be produced and stored so as to reduce degradation of the antioxidant/reducing agent and thereby enable stable nitric oxide generation.

### BACKGROUND

### Technical Field

Disclosed herein are materials, devices, methods, and systems, such as therapeutic compositions, wound care materials, their uses, and methods of treatment therewith. In some examples, the materials, devices, and systems described herein comprise a wound dressing configured for nitric oxide (NO) delivery and/or the delivery of other actives.

### Description of the Related Art

Nitric oxide (NO) is a well-known molecule with multiple biological functions. For example, nitric oxide influences blood vessel vasodilation, stimulates angiogenesis, influences the host immune response, and demonstrates potent, broad spectrum antimicrobial activity and anti-biofilm activity. Due to these multiple roles, NO demonstrates a potent effect on tissue and increased amounts of NO may support the acceleration of healing in wounds, particularly chronic wounds.

Additionally, diabetic patients often have lower levels of nitric oxide as compared to healthy patients, and diminished supply of nitric oxide in diabetic patients is a compounding factor in a healing chronic ulcer. Diminished supply of nitric oxide may lead to vascular damage, such as endothelial dysfunction and vascular inflammation. Vascular damage may also lead to decreased blood flow to the extremities, thereby potentially causing the diabetic patient to be more likely to develop neuropathy and non-healing ulcers, and to be at a greater risk for lower limb amputation.

Consequently, there is a need for improved mechanisms of delivering an effective dose of nitric oxide to a wound. Under normal conditions, nitric oxide (NO), a free radical, is short-lived and converted to a more stable chemical species within seconds of production. Thus, for example, if gaseous nitric oxide contacts air, the gaseous nitric oxide will be rapidly oxidized to generate nitrogen dioxide (NO₂). Accordingly, it may be difficult to maintain high concentrations of nitric oxide within a wound dressing or other similar structure for a prolonged period of time. Therefore, a device or a wound dressing having one or more layers containing more stable compositions may effectively generate nitric oxide over time upon activation, for the stable and sustained delivery of nitric oxide to biological tissues. Of particular interest are mechanisms of delivering nitric oxide in combination with use of a wound dressing, particularly a negative pressure wound dressing and/or while undergoing negative pressure wound therapy and/or other appropriate therapies.

### SUMMARY

Embodiments of the present disclosure relate to materials, devices, methods, and systems for wound treatment. Some disclosed embodiments relate to materials, devices, methods, and systems for delivering nitric oxide to a wound. It will be understood by one of skill in the art that application of the materials, devices, methods, and systems described herein are not limited to a particular tissue or a particular injury.

In some embodiments, a wound dressing for treating a wound may comprise a cover layer configured to form a seal around a wound, an activator layer, a dry nitrite providing layer, the dry nitrite providing layer free or relatively free of liquid, and an acquisition distribution layer.

In certain embodiments, the wound dressing may further comprise a masking layer, the masking layer configured to at least partially limit visualization of the wound. The dry nitrite providing layer may comprise a nitrite salt. The nitrite salt may comprise sodium nitrite. The dry nitrite providing layer may further comprise an antioxidant/reducing agent. The reducing agent may be sodium iso-ascorbate. The nitrite salt and antioxidant/reducing agent of the dry nitrite providing layer may be colocalized on a mesh component. The nitrite salt and antioxidant/reducing agent may be colocalized on the mesh component by embedding a mixture of the nitrite salt and reducing agent within the mesh component. The activator layer may be positioned above the nitrite providing layer. In some embodiments, the nitrite providing layer may be positioned above the activator layer. The acquisition distribution layer may be positioned between the activator layer and the dry nitrite providing layer. The activator layer may comprise a hydrogel or a xerogel. The wound dressing may comprise a second dry nitrite providing layer. The wound dressing may be configured to generate nitric oxide when the wound dressing is placed over a wound. In some embodiments, the wound dressing may be configured to not generate nitric oxide prior to placement over a wound.

In some aspects, a wound treatment apparatus for treating a wound is provided. In some embodiments, the wound treatment apparatus may comprise a nitrite providing layer comprising a mesh component. In some embodiments, the mesh component may comprise a colocalized nitrite salt and antioxidant/reducing agent. In some embodiments, the wound treatment apparatus may comprise an activator layer comprising a hydrogel. In some embodiments, the activator layer may be configured to donate a proton to the nitrite providing layer to produce nitric oxide. In some embodiments, the nitrite salt and antioxidant/reducing agent may be embedded within the mesh component. In some embodiments, the nitrite salt and antioxidant/reducing agent may be solubilized, and/or suspended in an anhydrous carrier prior to embedding within the mesh component. In some embodiments, the anhydrous carrier is soluble or miscible in water. In some embodiments, the anhydrous water-miscible carrier may be selected from the group consisting of polyethylene glycol (PEG)200, PEG400, PEG600, blends of PEG with different molecular weights, glycerol, triacetin, acetaldehyde, acetic acid, acetone, acetonitrile, 1,2-Butanediol, 1,3-Butanediol, 2-Butoxyethanol, butyric acid, diethanolamine, diethylenetriamine, dimethylformamide, dimethoxyethane, dimethyl sulfoxide, 1,4-Dioxane, ethanol, ethylamine, ethylene glycol, formic acid, furfuryl alcohol, methanol, methyl diethanolamine, methyl isocyanide, N-Methyl-2-pyrrolidone, 1-Propanediol, 1,3-Propanediol, 1,5-Pentanediol, 1-Propanol, 2-Propanol, propanoic acid, propylene glycol, pyridine, tetrahydrofuran, triethylene glycol. In some embodiments, the anhydrous water-miscible carrier may further comprise a surfactant. In some embodiments, the mesh component of the nitrite providing layer may be stored in a dry state. In some embodiments, the antioxidant/reducing agent colocalized with the nitrite salt in the mesh component of the nitrite providing layer may have greater stability than an antioxidant/reducing agent not stored in a dry state. In some embodiments, the antioxidant/reducing agent may demonstrate greater stability following sterilization compared to the stability of an antioxidant/reducing agent within a sterilized wound dressing wherein the antioxidant/reducing agent is not stored in a dry state. In some embodiments, the wound treatment apparatus may be configured to develop less coloration than a wound treatment apparatus wherein the antioxidant/reducing agent is not stored in a dry state. In some embodiments, the mesh may comprise a coated polypropylene mesh. In some embodiments, the activator layer may comprise a composite comprising a gelling fiber substrate loaded with an acidic hydrogel. In some embodiments, the hydrogel may further comprise aqueous Sodium 2-Acrylamido-2-methyl-1-propanesulfonate solution and 2-Acrylamido-2-methyl-1-propanesulfonic acid. In some embodiments, the wound treatment apparatus may further comprise an acquisition distribution layer. In some embodiments, the wound treatment apparatus may further comprise a cover layer configured to form a seal around the wound. In some embodiments, the cover layer may be moisture vapor permeable. In some embodiments, the wound treatment apparatus may further comprise a masking layer, the masking layer may be configured to at least partially limit visualization of the wound. In some embodiments, the nitrite salt may be sodium nitrite. In some embodiments, the antioxidant/reducing agent may be sodium iso-ascorbate, iron sulfate, or a derivative of vitamin C. Derivatives of vitamin C may include L-Ascorbic acid, sodium ascorbyl phosphate, magnesium ascorbyl phosphate, ascorbyl glucoside, tetrahexyldecyl ascorbate, ethylated L-asorbic acid, or any suitable vitamin C derivative. In some embodiments, the mesh component comprises a 2.5×2.5 cm mesh loaded with any amount between 1-5 mg of each of a nitrite salt and an antioxidant/reducing agent, wherein the nitrite salt and antioxidant/reducing agent are suspended in a suitable carrier.

In some aspects, a method for treating a wound is provided. In some embodiments, the method may comprise applying a wound dressing to the wound. In some embodiments, the wound dressing may comprise a nitrite providing layer comprising a mesh component. In some embodiments, the wound dressing may comprise a nitrite providing layer comprising a mesh component. In some embodiments, the mesh component may comprise a colocalized nitrite salt and antioxidant/reducing agent. In some embodiments, the wound dressing may comprise an activator layer comprising a hydrogel. In some embodiments, the activator layer may be configured to donate a proton to the nitrite providing layer to produce nitric oxide. In some embodiments, the nitrite salt and antioxidant/reducing agent may be embedded within the mesh component. In some embodiments, the nitrite salt and antioxidant/reducing agent may be solubilized, and/or suspended in an anhydrous carrier prior to embedding within the mesh component. In some embodiments, the anhydrous carrier is soluble or miscible in water. In some embodiments, the anhydrous water-miscible carrier may be selected from the group consisting of polyethylene glycol (PEG)200, PEG400, PEG600, blends of PEG with different molecular weights, glycerol, triacetin, acetaldehyde, acetic acid, acetone, acetonitrile, 1,2-Butanediol, 1,3-Butanediol, 2-Butoxyethanol, butyric acid, diethanolamine, diethylenetriamine, dimethylformamide, dimethoxyethane, dimethyl sulfoxide, 1,4-Dioxane, ethanol, ethylamine, ethylene glycol, formic acid, furfuryl alcohol, methanol, methyl diethanolamine, methyl isocyanide, N-Methyl-2-pyrrolidone, 1-Propanediol, 1,3-Propanediol, 1,5-Pentanediol, 1-Propanol, 2-Propanol, propanoic acid, propylene glycol, pyridine, tetrahydrofuran, triethylene glycol. In some embodiments, the anhydrous water-miscible carrier may further comprise a surfactant. In some embodiments, the mesh component of the nitrite providing layer may be stored in a dry state. In some embodiments, the antioxidant/reducing agent colocalized with the nitrite salt in the mesh component of the nitrite providing layer may have greater stability than an antioxidant/reducing agent not stored in a dry state. In some embodiments, the antioxidant/reducing agent may demonstrate greater stability following sterilization compared to the stability of an antioxidant/reducing agent within a sterilized wound dressing wherein the antioxidant/reducing agent is not stored in a dry state. In some embodiments, the wound dressing may be configured to develop less coloration than a wound treatment apparatus wherein the antioxidant/reducing agent is not stored in a dry state. In some embodiments, the mesh may comprise a coated polypropylene mesh. In some embodiments, the activator layer may comprise a composite comprising a gelling fiber substrate loaded with an acidic hydrogel. In some embodiments, the hydrogel may further comprise aqueous Sodium 2-Acrylamido-2-methyl-1-propanesulfonate solution and 2-Acrylamido-2-methyl-1-propanesulfonic acid. In some embodiments, the wound dressing may further comprise an acquisition distribution layer. In some embodiments, the wound dressing may further comprise a cover layer configured to form a seal around the wound. In some embodiments, the cover layer may be moisture vapor permeable. In some embodiments, the wound dressing may further comprise a masking layer, the masking layer may be configured to at least partially limit visualization of the wound. In some embodiments, the nitrite salt may be sodium nitrite. In some embodiments, the antioxidant/reducing agent may be sodium iso-ascorbate, iron sulfate, or a derivative of vitamin C. In some embodiments, the mesh component comprises a 2.5×2.5 cm mesh loaded with any amount between 1-5 mg of each of a nitrite salt and an antioxidant/reducing agent, wherein the nitrite salt and antioxidant/reducing agent are suspended in a suitable carrier.

Alternative or additional embodiments described herein provide a composition comprising one or more of the features of the foregoing description or of any description elsewhere herein.

Alternative or additional embodiments described herein provide a wound contact layer comprising one or more of the features of the foregoing description or of any description elsewhere herein.

Alternative or additional embodiments described herein provide a wound dressing comprising one or more of the features of the foregoing description or of any description elsewhere herein.

Alternative or additional embodiments described herein provide a wound treatment system comprising one or more of the features of the foregoing description or of any description elsewhere herein.

Alternative or additional embodiments described herein provide a method of treating a wound comprising one or more of the features of the foregoing description or of any description elsewhere herein.

### BRIEF DESCRIPTION OF THE DRAWINGS

FIG. 1 is a schematic diagram of an example of a negative pressure wound therapy system;

FIG. 2A illustrates an embodiment of a negative pressure wound treatment system employing a pump, a flexible fluidic connector and a wound dressing capable of absorbing and storing wound exudate;

FIG. 2B illustrates an embodiment of a negative pressure wound treatment system employing a flexible fluidic connector and a wound dressing capable of absorbing and storing wound exudate;

FIG. 2C illustrates a cross section of an embodiment of a fluidic connector connected to a wound dressing;

FIG. 2D illustrates a cross-section of an embodiment of a wound dressing;

FIGS. 3A-3D illustrate embodiments of wound dressings capable of absorbing and storing wound exudate to be used without negative pressure;

FIG. 3E illustrates a cross section of an embodiment of a wound dressing capable of absorbing and storing wound exudate to be used without negative pressure;

FIG. 4 is an exploded view of an embodiment of a wound dressing which can generate nitric oxide;

FIG. 5 is a cross sectional view of the wound dressing of FIG. 4;

FIG. 6 illustrates an example of a chemiluminescence experimental protocol equipment setup;

FIG. 7 illustrates a negative pressure and nitric oxide delivery experiment;

FIG. 8A depicts an example of chemiluminescence experimental results for a sodium nitrate mesh;

FIG. 8B depicts an example of chemiluminescence experimental results for a full dressing design with a pull-out tab and self-sealing borders;

FIG. 8C depicts an example of chemiluminescence experimental results for a dressing containing a degradable film;

FIG. 9 depicts an example of a graph displaying peak NO and NO₂ outputs for acrylic adhesive containing hydrogels;

FIGS. 10A-10D depict examples of chemiluminescence experimental results for nitric oxide dressing;

FIGS. 11A-11E depicts embodiments of a wound dressing configured to generate nitric oxide;

FIG. 12 illustrates an embodiment of a hydrogel-based wound dressing system;

FIG. 13 illustrates an embodiment of a hydrogel-based wound dressing system;

FIG. 14 depicts an example process of hydrogel production;

FIG. 15 depicts an example of chemiluminescence experimental results for a representative acidic AMPS-based hydrogel without sodium iso-ascorbate when contacted with a polypropylene mesh soaked in aqueous sodium nitrite;

FIG. 16 depicts an example of chemiluminescence results for an acidic hydrogel with 1.0 SA contacted a suspension generated by dispersing 0.1 g sodium nitrite and 0.1 g sodium iso-ascorbate in 2 g of PEG400. A 1.0 SA gel may be made of the following components (parts by weight of a 100 g batch): 63.32 g sodium AMPS; 0.0323 g piperazine diacrylamide; 5.436 g AMPS (H+); 31.19 g glycerol and 0.01629 g 2-hydroxy-2-methylpropiophenone. The upper plotted line is NO₂, while the bottom line is NO.;

FIG. 17A illustrates an experimental set up for an evaluation of the antimicrobial activity of a dressing composed of an IV3000 top layer, a hydrogel/DURAFIBER composite with 1.0 SA or 0.7 SA, and a polypropylene (pp) mesh embedded with a PEG400/sodium nitrite/sodium iso-ascorbate suspension. A 0.7 SA gel may be made of the following components (parts by weight of a 100 g batch): 65.28 g sodium AMPS; 0.0317 g piperazine diacrylamide; 3.923 g AMPS (H+); 30.74 g glycerol and 0.01756 g 2-hydroxy-2-methylpropiophenone. The top-most plotted line is NOX, the middle plotted line is NO, and the bottom line is NO₂;

FIG. 17B illustrates an overhead view of the experimental set up of the evaluation in FIG. 17A;

FIG. 17C depicts the results in terms of reduction from inoculum control for the evaluation illustrated in FIGS. 17A-17B;

FIGS. 18A-18D show chemiluminescence results for 3×3 cm composites of DURAFIBER loaded with ~6 g 1.0 SA acidic hydrogel and contacted with 2.5×2.5 PP mesh embedded with 50 μL of a suspension generated by dispersing 0.1 g sodium nitrite and 0.1 g sodium iso-ascorbate in various anhydrous water miscible carriers;

FIG. 18A shows nitric oxide production where PEG400 was used as the anhydrous water miscible carrier. The top-most plotted line is NOX, the middle line is NO, and the bottom is NO₂;

FIG. 18B shows the results when PEG600 was used as the anhydrous water miscible carrier. The top-most plotted line is NOX, the middle is NO, and the bottom is NO₂;

FIG. 18C shows the results when a 50/50 w/w blend of PEG300 and PEG1500 (FLEX) was used as the anhydrous water miscible carrier. The top-most plotted line is NOX, the middle line is NO, and the bottom line is NO₂;

FIG. 18D shows the results when 41/1 w/w blend of PEG400 and PEG4000 (SORB) was used as the anhydrous water miscible carrier. The top-most plotted line is NOX, the middle line is NO, and the bottom line is NO₂;

FIG. 18E combines the results of FIGS. 18A-18D on a single graph.

### DETAILED DESCRIPTION

### Overview

Embodiments described herein relate to materials, apparatuses, methods, and systems that incorporate, or comprise, or utilize one or more compositions and/or materials that effectively generate gases (e.g. nitric oxide) over time upon activation. Embodiments herein may be directed toward a device and/or a wound dressing having one or more layers containing compositions and/or materials that effectively generate nitric oxide over time upon activation. For example, one or more nitric oxide generating layers may include a nitrite delivery layer which contains nitrite salts and can release nitrite ions, such that the nitrite ions can generate nitric oxide upon reaction with acids.

In some embodiments, the one or more nitric oxide generating layers can further include an acidic-group-providing layer in addition to the nitrite delivery layer. The one or more nitric oxide generating layers may be utilized as a stand-alone component for separately positioning at a wound site, or may be incorporated into any number of multi-layer wound dressings and wound treatment apparatuses, such as described herein below with respect to FIGS. 1 through 18E. Embodiments of the present disclosure are generally applicable to use under ambient conditions, in negative pressure or reduced pressure therapy systems, or in compression therapy systems.

Some of the preferred embodiments described herein incorporate, or comprise, or utilize one or more nitric oxide generating layers. Such one or more nitric oxide generating layers may possess one or more of the following functional features: inflammation-related activities, blood flow-related activities, antimicrobial, anti-planktonic and anti-biofilm activities, ease of application or/and removal as one piece, cuttability/tearability, conformability to the three-dimensional contour of a wound surface, durability to wear, compatibility with negative pressure wound therapy or/and compression wound therapy, exudate management, capability of facilitating autolytic debridement of wounds, capability of promoting wound healing, and self-indication of compositional or functional changes. The antimicrobial activities, such as in vitro antimicrobial activities, can include one or more of the following: broad-spectrum antimicrobial activity, anti-biofilm activity, rapid speed of kill against microorganisms, sustained kill against microorganisms; and the microorganisms can include one or more of the following: Gram-negative bacteria, Gram-positive bacteria, fungi, yeasts, viruses, algae, archaea and protozoa.

Certain preferred embodiments described herein provide a wound treatment system. Such a wound treatment system may comprise nitric oxide generating layers, configured to be sized for positioning over a wound and/or the periwound area. One of skill in the art will understand that when an apparatus/dressing/layer is described as being placed on or over a wound, such an apparatus/dressing/layer may extend over and treat the periwound area. In some instances, stimulation of the periwound area and/or the wound edge may play a role in initiating the wound healing process, and the wound healing process can be activated through the delivery of nitric oxide to the periwound area and/or the wound edge. The delivery of nitric oxide to the periwound area and/or the wound edge may target, for example epithelial cell activity to promote migration of epithelial tongue; vasodilation of the microcirculation in the skin surrounding the wound to promote profusion by providing oxygen and nutrients; and neo-angiogenesis to promote granulation tissue formation. The wound treatment systems described herein may further comprise a secondary wound dressing configured to be separately positioned over the nitric oxide generating layers. The nitric oxide generating layers may have an adhesive adhered to the lower surface; and the adhesive can be configured such that the nitric oxide generating layers may be placed in proximity to the wound. The secondary wound dressing, if used, may adhere to skin surrounding the wound and may have the same size or may be larger than the nitric oxide generating layers, such that the nitric oxide generating layers will touch or be placed in proximity to the wound and/or the periwound area. The secondary wound dressing can be alternatively or additionally configured to form a seal to skin surrounding the wound so that the nitric oxide generating layers will touch or be placed in proximity to the wound. The wound treatment system may further comprise a source of negative pressure configured to supply negative pressure through the secondary wound dressing and through the wound contact layer to the wound.

Certain other preferred embodiments described herein provide a multi-layered wound dressing, such as described herein the specification with respect to FIGS. 1 through 13. Such a multi-layered wound dressing may incorporate the one or more nitric oxide generating layers as component layers thereof or, alternatively, may comprise a composite or laminate including the one or more nitric oxide generating layers as part of one of the component layers thereof. The multi-layered wound dressing may comprise: nitric oxide generating layers as described above or described elsewhere herein; a transmission layer and/or absorbent layer over/under the one or more nitric oxide generating layers; a wound contact layer under the one or more nitric oxide generating layers; and a cover layer over the transmission layer and/or absorbent layer. The wound dressing may further comprise a negative pressure port positioned on or above the cover layer. The one or more nitric oxide generating layers may have a perimeter shape that is substantially the same as a perimeter shape of the cover layer. Alternatively, the one or more nitric oxide generating layers may have a perimeter shape that is smaller than a perimeter shape of the cover layer.

One of skill in the art will understand that nitric oxide generating compositions, such as any disclosed herein this “Overview” section or elsewhere in the specification, may be loaded within the one or more nitric oxide generating layers in any suitable form, such as via adsorption, absorption, chemical and/or physical attachment entanglement, and/or via powder form. One of skill in the art will further understand that reactive compositions, such as any disclosed herein this section or elsewhere in the specification may be incorporated into any suitable absorbent layer disclosed herein this section or elsewhere in the specification by any suitable means, and/or any suitable transmission layer disclosed herein this section or elsewhere in the specification, and/or any foam layer disclosed herein this section or elsewhere in the specification.

In certain embodiments, the wound treatment systems and multi-layered wound dressings disclosed above or disclosed elsewhere herein the specification may incorporate or comprise nitric oxide generating layers. As described herein this section or elsewhere in the specification, particularly below, the nitric oxide generating layers may be configured to be activated to release nitric oxide. At least a portion of the released nitric oxide may be released, for example by diffusion. To facilitate release and diffusion of nitric oxide, the nitric oxide generating layers may be placed proximate to the wound.

Some preferred embodiments described herein the specification provide a method to treat a wound, intact tissue, or other suitable location. Such a method may include placing nitric oxide generating layers, either separately or by placing a multi-layered wound dressing having nitric oxide generating layers, over the wound. The method may comprise adhering the separate nitric oxide generating layers and/or the multi-layer wound dressing having nitric oxide generating layers to healthy skin around the wound. Such a method may further comprise one or more of the following steps: A further wound dressing can be placed over the separate nitric oxide generating layers or multi-layered wound dressing having the nitric oxide generating layers that is placed over the wound. Wound exudate, or any moist or aqueous medium other than wound exudate, may be provided to reach and/or touch the nitric oxide generating layers. Wound exudate, or any moist or aqueous medium other than wound exudate may be diffused or wicked into the wound dressing incorporating the nitric oxide generating layers or into a wound dressing provided over the nitric oxide generating layers. Negative pressure may be applied to the separate nitric oxide generating layers or multi-layered wound dressing having the nitric oxide generating layers, such that wound exudate is suctioned into the nitric oxide generating layers directly, or into the wound dressing incorporating the nitric oxide generating layers, or into a wound dressing provided over the nitric oxide generating layers.

One of skill in the art will understand that wound dressings, devices and systems disclosed herein this “Overview” section or elsewhere in the specification may include one or more layers, compositions, materials or components that generate gases other than nitric oxide in addition to or in place of the nitric oxide generating layers, compositions or materials. For example, a wound dressing or a device can include one or more layers that effectively generate vasodilatory agents, such as carbon monoxide or hydrogen sulfide, over time upon activation.

One of skill in the art will further understand that carbon monoxide and/or hydrogen sulfide may be used in place of a nitric oxide delivery element (such as a layer) or in combination with a nitric oxide delivery element (such as a layer) where suitable. Further details regarding generation and delivery of carbon monoxide and/or hydrogen sulfide may be found in chapter six of the text Inorganic and Organometallic Transition Metal Complexes with Biological Molecules and Living Cells, ISBN 978-0-12-803814-7, which is hereby incorporated by reference. For example, hydrogen sulfide may be generated from elements/layers that contain cleavable/releasable hydrogen sulfide, diallyl thiosulfinate, GYY4137, S-Mesalamine ATB-429, S-Naproxen ATB-346, S-Diclofenac ATB-337/ACS-15. For example, carbon monoxide may be generated from elements/layers that provide complexes of carbon monoxide bound to suitable metals such as chromium, molybdenum, tungsten, manganese, rhenium, iron, ruthenium, cobalt, rhodium, and iridium. Such complexes may be enzymatically triggered to release carbon monoxide, photo-cleavable, and/or responsive to interaction with a suitable ligand to induce release of carbon monoxide.

### Method of Treating a Wound

Some preferred embodiments described herein the specification provide a method of treating a wound, intact tissue, or other suitable location. Such a method may include placing one or more nitric oxide generating layers, either separately or by placing a multi-layered wound dressing having one or more nitric oxide generating layers over the wound. The method may comprise adhering the separate one or more nitric oxide generating layers and/or the multi-layer wound dressing having one or more nitric oxide generating layers to healthy skin around the wound, such as the periwound area. The method may further comprise one or more of the following steps: A further wound dressing can be placed over the separate one or more nitric oxide generating layers or multi-layered wound dressing having the one or more nitric oxide generating layers that is placed over the wound. Wound exudate, or any moist or aqueous medium other than wound exudate, may be provided to reach and/or touch the one or more nitric oxide generating layers. Wound exudate, or any moist or aqueous medium other than wound exudate may be diffused or wicked into the wound dressing incorporating the one or more nitric oxide generating layers or into a wound dressing provided over the one or more nitric oxide generating layers. Negative pressure may be applied to the separate one or more nitric oxide generating layers or multi-layered wound dressing having the one or more nitric oxide generating layers, as described in the following “Negative Pressure Wound Therapy (NPWT) Systems” section or described elsewhere herein the specification, such that wound exudate is suctioned into the one or more nitric oxide generating layers directly, or into the wound dressing incorporating the one or more nitric oxide generating layers, or into a wound dressing provided over the one or more nitric oxide generating layers.

The method of treating a wound, intact tissue, or other suitable location as described above or described elsewhere herein may further comprise delivering negative pressure through the wound contact layer to the wound, as described in the following “Negative Pressure Wound Therapy (NPWT) Systems” section or described elsewhere herein the specification. The wound contact layer may substantially maintain the negative pressure delivered for at least about 24 hours, or for at least about 48 hours, or for at least about 72 hours. Alternatively, the method of treating a wound, intact tissue, or other suitable location may comprise applying compression (positive) pressure through the wound contact layer to the wound. Alternatively, the method may comprise altering ambient pressure, negative pressure and compression pressure in a programmable manner through the wound contact layer to the wound.

In some embodiments, the method of treating a wound, intact tissue, or other suitable location may comprise using the wound contact layer, or the wound treatment system or wound dressing that comprises the wound contact layer, under ambient conditions not in connection with a negative pressure wound therapy system as described above, or described elsewhere herein.

In some embodiments, a method of treating a wound, intact tissue, or other suitable location may reduce the wound bioburden, for example, at least in vitro, by reducing the numbers (CFU/sample) of viable microorganisms within the first 4 hours after the application wound contact layer. In some examples, the numbers of viable microorganisms may be reduced by four log or more, 48 to 72 hours after positioning the wound dressing in contact with the microorganisms.

### Negative Pressure Wound Therapy (NPWT) Systems

It will be understood that embodiments of the present disclosure are generally applicable to, but not limited to, use in topical negative pressure (“TNP”) therapy systems. Briefly, negative pressure wound therapy assists in the closure and healing of many forms of “hard to heal” wounds by reducing tissue oedema; encouraging blood flow and granular tissue formation; removing excess exudate and may reduce bacterial load (and thus infection risk). In addition, the therapy allows for less disturbance of a wound leading to more rapid healing. TNP therapy systems may also assist on the healing of surgically closed wounds by removing fluid and by helping to stabilize the tissue in the apposed position of closure. A further beneficial use of TNP therapy can be found in grafts and flaps where removal of excess fluid is important and close proximity of the graft to tissue is required in order to ensure tissue viability.

As is used herein, reduced or negative pressure levels, such as −X mmHg, represent pressure levels relative to normal ambient atmospheric pressure, which can correspond to 760 mmHg (or 1 atm, 29.93 inHg, 101.325 kPa, 14.696 psi, etc.). Accordingly, a negative pressure value of −X mmHg reflects absolute pressure that is X mmHg below 760 mmHg or, in other words, an absolute pressure of (760-X) mmHg. In addition, negative pressure that is “less” or “smaller” than X mmHg corresponds to pressure that is closer to atmospheric pressure (e.g., −40 mmHg is less than-60 mmHg). Negative pressure that is “more” or “greater” than −X mmHg corresponds to pressure that is further from atmospheric pressure (e.g., −80 mmHg is more than −60 mmHg). In some embodiments, local ambient atmospheric pressure is used as a reference point, and such local atmospheric pressure may not necessarily be, for example, 760 mmHg.

The negative pressure range for some embodiments of the present disclosure can be approximately −80 mmHg, or between about −20 mmHg and about −200 mmHg. Note that these pressures are relative to normal ambient atmospheric pressure, which can be 760 mmHg. Thus, −200 mmHg would be about 560 mmHg in practical terms. In some embodiments, the pressure range can be between about −40 mmHg and about −150 mmHg. Alternatively, a pressure range of up to −75 mmHg, up to −80 mmHg or over −80 mmHg can be used. Also in other embodiments a pressure range of below −75 mmHg can be used. Alternatively, a pressure range of over approximately −100 mmHg, or even −150 mmHg, can be supplied by the negative pressure apparatus.

In some embodiments of wound closure devices described herein, increased wound contraction can lead to increased tissue expansion in the surrounding wound tissue. This effect may be increased by varying the force applied to the tissue, for example by varying the negative pressure applied to the wound over time, possibly in conjunction with increased tensile forces applied to the wound via embodiments of the wound closure devices. In some embodiments, negative pressure may be varied over time for example using a sinusoidal wave, square wave, or in synchronization with one or more patient physiological indices (e.g., heartbeat). Examples of such applications where additional disclosure relating to the preceding may be found include U.S. Pat. No. 8,235,955, titled “Wound treatment apparatus and method,” issued on Aug. 7, 2012; and U.S. Pat. No. 7,753,894, titled “Wound cleansing apparatus with stress,” issued Jul. 13, 2010. The disclosures of both of these patents are hereby incorporated by reference in their entirety.

Embodiments of the wound dressings, wound dressing components, wound treatment apparatuses and methods described herein may also be used in combination or in addition to those described in International Application No. PCT/IB2013/001469, filed May 22, 2013, published as WO 2013/175306 A2 on Nov. 28, 2013, titled “APPARATUSES AND METHODS FOR NEGATIVE PRESSURE WOUND THERAPY,” International Application No. PCT/IB2013/002060, filed on Jul. 31, 2013, published as WO2014/020440, entitled “WOUND DRESSING,” the disclosures of which are hereby incorporated by reference in their entireties. Embodiments of the wound dressings, wound treatment apparatuses and methods described herein may also be used in combination or in addition to those described in U.S. Pat. No. 9,061,095, titled “WOUND DRESSING AND METHOD OF USE,” issued on Jun. 23, 2015; and U.S. Application Publication No. 2016/0339158, titled “FLUIDIC CONNECTOR FOR NEGATIVE PRESSURE WOUND THERAPY,” published on Nov. 24, 2016, the disclosures of which are hereby incorporated by reference in its entirety, including further details relating to embodiments of wound dressings, the wound dressing components and principles, and the materials used for the wound dressings.

Additionally, some embodiments related to TNP wound treatment comprising a wound dressing in combination with a pump or associated electronics described herein may also be used in combination or in addition to those described in International Publication No. WO 2016/174048 A1, entitled “REDUCED PRESSURE APPARATUSES”, published on Nov. 3, 2016, the entirety of which is hereby incorporated by reference. In some of these embodiments, the pump or associate electronic components may be integrated into the wound dressing to provide a single article to be applied to the wound.

### Multi-Layered Wound Dressings for NPWT

FIG. 1 illustrates an example of a negative pressure wound therapy system 700. The system includes a wound cavity 710 covered by a wound dressing 720, which can be a dressing according to any of the examples described herein. The dressing 720 can be positioned on, inside, over, or around the wound cavity 710 and further seal the wound cavity so that negative pressure can be maintained in the wound cavity. For example, a film layer of the wound dressing 720 can provide substantially fluid impermeable seal over the wound cavity 710. In some embodiments, a wound filler, such as a layer of foam or gauze, may be utilized to pack the wound. The wound filler may include one or more nitric oxide generating layers (e.g. a nitrite delivery layer, an acidic-group providing layer) as described herein this section or elsewhere in the specification. For example, in a traditional negative pressure wound therapy system utilizing foam or gauze, such as the Smith & Nephew RENASYS Negative Pressure Wound Therapy System utilizing foam (RENASYS-F) or gauze (RENASYS-G), the foam or gauze may be supplemented with nitric oxide generating layers as described above. When supplementing a foam or gauze layer or other wound packing material, the one or more nitric oxide generating layers may either be separately inserted into the wound or may be pre-attached with the wound packing material for insertion into the wound.

A single or multi lumen tube or conduit 740 connects the wound dressing 720 with a negative pressure device 750 configured to supply reduced pressure. The negative pressure device 750 includes a negative pressure source. The negative pressure device 750 can be a canisterless device (meaning that exudate is collected in the wound dressing and/or is transferred via the tube 740 for collection to another location). In some embodiments, the negative pressure device 750 can be configured to include or support a canister. Additionally, in any of the embodiments disclosed herein, the negative pressure device 750 can be fully or partially embedded in, mounted to, or supported by the wound dressing 720.

The conduit 740 can be any suitable article configured to provide at least a substantially sealed fluid flow path or pathway between the negative pressure device 750 and the wound cavity 710 so as to supply reduced pressure to the wound cavity. The conduit 740 can be formed from polyurethane, PVC, nylon, polyethylene, silicone, or any other suitable rigid or flexible material. In some embodiments, the wound dressing 720 can have a port configured to receive an end of the conduit 740. For example, a port can include a hole in the film layer. In some embodiments, the conduit 740 can otherwise pass through and/or under a film layer of the wound dressing 720 to supply reduced pressure to the wound cavity 710 so as to maintain a desired level of reduced pressure in the wound cavity. In some embodiments, at least a part of the conduit 740 is integral with or attached to the wound dressing 720.

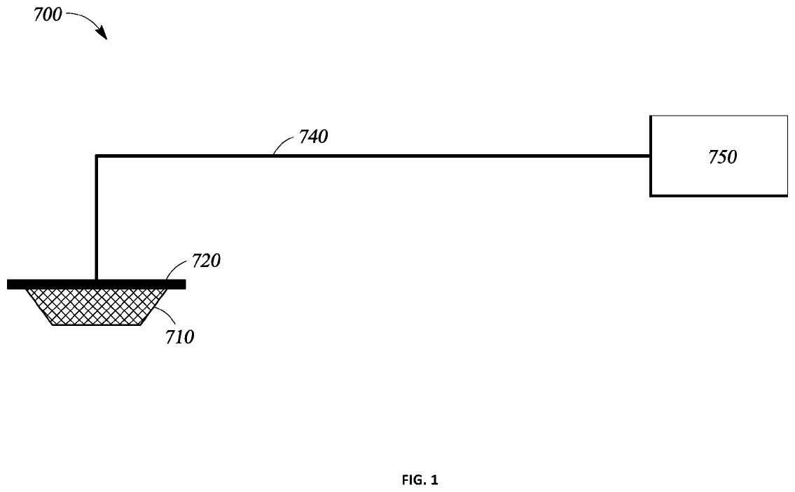

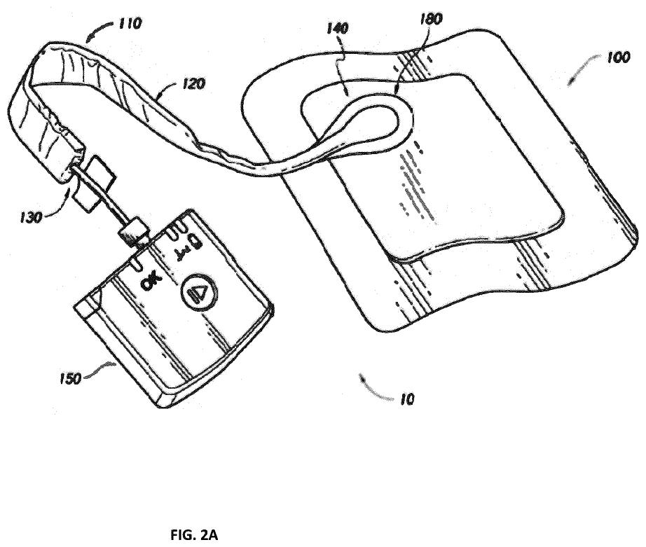

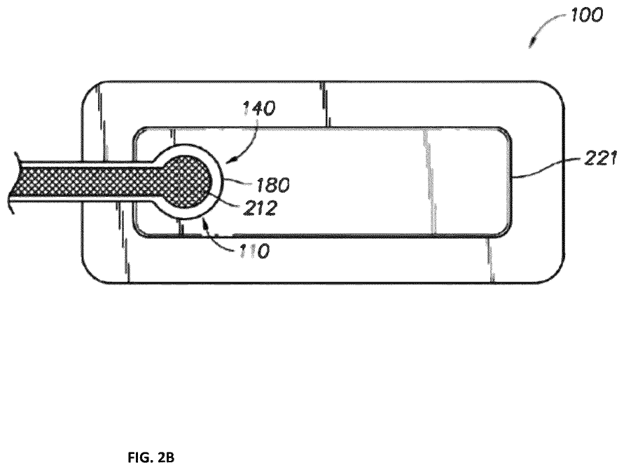

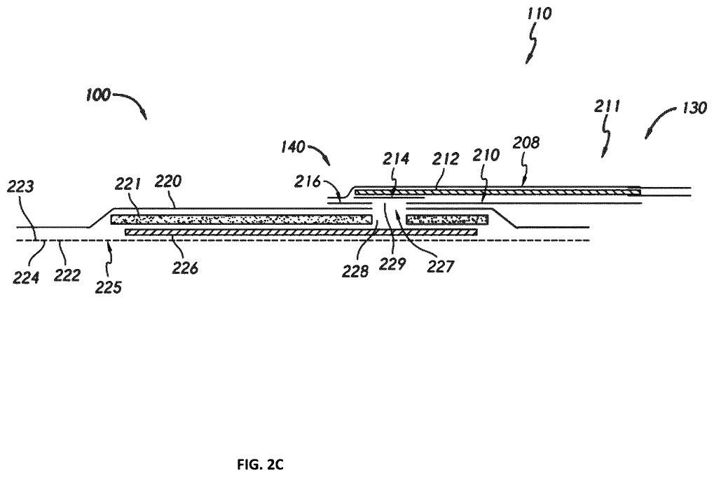

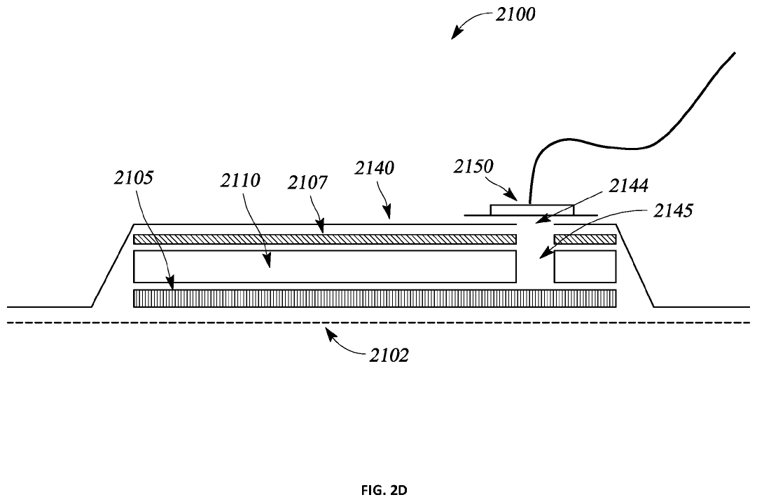

FIG. 2A illustrates an embodiment of a negative pressure wound treatment system 10 employing a wound dressing 100 in conjunction with a fluidic connector 110. Additional examples related to negative pressure wound treatment comprising a wound dressing in combination with a pump as described herein may also be used in combination or in addition to those described in U.S. Pat. No. 9,061,095, which is incorporated by reference in its entirety. Here, the fluidic connector 110 may comprise an elongate conduit, more preferably a bridge 120 having a proximal end 130 and a distal end 140, and an applicator 180 at the distal end 140 of the bridge 120. The system 10 may include a source of negative pressure such as a pump or negative pressure unit 150 capable of supplying negative pressure. The pump may comprise a canister or other container for the storage of wound exudates and other fluids that may be removed from the wound. A canister or container may also be provided separate from the pump. In some embodiments, the pump 150 can be a canisterless pump such as the PICO™ pump, as sold by Smith & Nephew. The pump 150 may be connected to the bridge 120 via a tube, or the pump 150 may be connected directly to the bridge 120. In use, the dressing 100 is placed over a suitably-prepared wound, which may in some cases be filled with a wound packing material such as foam or gauze as described above. The applicator 180 of the fluidic connector 110 has a sealing surface that is placed over an aperture in the dressing 100 and is sealed to the top surface of the dressing 100. Either before, during, or after connection of the fluidic connector 110 to the dressing 100, the pump 150 is connected via the tube to a coupling of the tube, or is connected directly to the bridge 120. The pump is then activated, thereby supplying negative pressure to the wound. Application of negative pressure may be applied until a desired level of healing of the wound is achieved.

As shown in FIG. 2B, the fluidic connector 110 preferably comprises an enlarged distal end, or head 140 that is in fluidic communication with the dressing 100 as will be described in further detail below. In one embodiment, the enlarged distal end has a round or circular shape. The head 140 is illustrated here as being positioned near an edge of the dressing 100, but may also be positioned at any location on the dressing. For example, some embodiments may provide for a centrally or off-centered location not on or near an edge or corner of the dressing 100. In some embodiments, the dressing 10 may comprise two or more fluidic connectors 110, each comprising one or more heads 140, in fluidic communication therewith. In a preferred embodiment, the head 140 may measure 30 mm along its widest edge. The head 140 forms at least in part the applicator 180, described above, that is configured to seal against a top surface of the wound dressing.

FIG. 2C illustrates a cross-section through a wound dressing 100 similar to the wound dressing 10 as described in International Patent Publication WO2013175306 A2, which is incorporated by reference in its entirety, along with fluidic connector 110. The wound dressing 100, which can alternatively be any wound dressing embodiment disclosed herein or any combination of features of any number of wound dressing embodiments disclosed herein, can be located over a wound site to be treated. The dressing 100 may be placed as to form a sealed cavity over the wound site. In a preferred embodiment, the dressing 100 comprises a top or cover layer, or backing layer 220 attached to an optional wound contact layer 222, both of which are described in greater detail below. These two layers 220, 222 are preferably joined or sealed together so as to define an interior space or chamber. This interior space or chamber may comprise additional structures that may be adapted to distribute or transmit negative pressure, store wound exudate and other fluids removed from the wound, and other functions which will be explained in greater detail below. Examples of such structures, described below, include a transmission layer 226 and an absorbent layer 221.

As used herein the upper layer, top layer, or layer above refers to a layer furthest from the surface of the skin or wound while the dressing is in use and positioned over the wound. Accordingly, the lower surface, lower layer, bottom layer, or layer below refers to the layer that is closest to the surface of the skin or wound while the dressing is in use and positioned over the wound.

As illustrated in FIG. 2C, the wound contact layer 222 can be a polyurethane layer or polyethylene layer or other flexible layer which is perforated, for example via a hot pin process, laser ablation process, ultrasound process or in some other way or otherwise made permeable to liquid and gas. The wound contact layer 222 has a lower surface 224 and an upper surface 223. The perforations 225 preferably comprise through holes in the wound contact layer 222 which enable fluid to flow through the layer 222. The wound contact layer 222 helps prevent tissue ingrowth into the other material of the wound dressing. Preferably, the perforations are small enough to meet this requirement while still allowing fluid to flow therethrough. For example, perforations formed as slits or holes having a size ranging from 0.025 mm to 1.2 mm are considered small enough to help prevent tissue ingrowth into the wound dressing while allowing wound exudate to flow into the dressing. In some configurations, the wound contact layer 222 may help maintain the integrity of the entire dressing 100 while also creating an air tight seal around the absorbent pad in order to maintain negative pressure at the wound.

Some embodiments of the wound contact layer 222 may also act as a carrier for an optional lower and upper adhesive layer (not shown). For example, a lower pressure sensitive adhesive may be provided on the lower surface 224 of the wound dressing 100 whilst an upper pressure sensitive adhesive layer may be provided on the upper surface 223 of the wound contact layer. The pressure sensitive adhesive, which may be a silicone, hot melt, hydrocolloid or acrylic based adhesive or other such adhesives, may be formed on both sides or optionally on a selected one or none of the sides of the wound contact layer. When a lower pressure sensitive adhesive layer is utilized, it may be helpful to adhere the wound dressing 100 to the skin around a wound site. In some embodiments, the wound contact layer may comprise perforated polyurethane film. The lower surface of the film may be provided with a silicone pressure sensitive adhesive and the upper surface may be provided with an acrylic pressure sensitive adhesive, which may help the dressing maintain its integrity. In some embodiments, a polyurethane film layer may be provided with an adhesive layer on both its upper surface and lower surface, and all three layers may be perforated together.

A transmission layer 226 can be located above the wound contact layer 222. In some embodiments, the transmission layer can be a porous material. As used herein the transmission layer can be referred to as a spacer layer and the terms can be used interchangeably to refer to the same component described herein. This transmission layer 226 allows transmission of fluid including liquid and gas away from a wound site into upper layers of the wound dressing. In particular, the transmission layer 226 preferably ensures that an open-air channel can be maintained to communicate negative pressure over the wound area even when the absorbent layer has absorbed substantial amounts of exudates. The layer 226 should preferably remain open under the typical pressures that will be applied during negative pressure wound therapy as described above, so that the whole wound site sees an equalized negative pressure. The layer 226 may be formed of a material having a three-dimensional structure. For example, a knitted or woven spacer fabric (for example Baltex 7970 weft knitted polyester) or a non-woven fabric could be used. The three-dimensional material can comprise a 3D spacer fabric material similar to the material described in International Publication WO 2013/175306 A2 and International Publication WO2014/020440, the disclosures of which are incorporated by reference in their entireties.

In certain embodiments, the wound dressing 100 may incorporate or comprise one or more nitric oxide generating layers (e.g. a nitrite delivery layer, an acidic-group providing layer) as described herein this section or elsewhere in the specification. One of skill in the art will understand that the wound dressing 100 may incorporate any of the one or more nitric oxide generating layers disclosed herein this section or elsewhere in the specification. One of skill in the art will also understand that the one or more nitric oxide generating layers may be incorporated as a whole component layer or a part of a component layer. In some embodiments, the one or more nitric oxide generating layers may be provided below the transmission layer 226. In some embodiments, the one or more nitric oxide generating layers may be provided above the wound contact layer 222. In certain embodiments, the one or more nitric oxide generating layers may replace the transmission layer 226, such that the one or more nitric oxide generating layers are provided between an absorbent layer 221 (described further below) and the wound contact layer 222. In some embodiments, the one or more nitric oxide generating layers can supplement or replace the absorbent layer 221. In some embodiments, the wound dressing 100 does not have the wound contact layer 222, and the one or more nitric oxide generating layers may be the lowermost layer of the wound dressing 100. The one or more nitric oxide generating layers may have same or substantially similar size and shape with the transmission layer 226 and/or the absorbent layer 221. In some embodiments, the one or more nitric oxide generating layers or components thereof (e.g., a nitrite providing layer as described herein) can be separate of the wound dressing 100. For example, the one or more nitric oxide generating layers or components thereof can be provided as a separate layer that can be placed on a wound, and the wound dressing 100 can be placed thereupon.

The one or more nitric oxide generating layers may be constructed to be flexible but stiff enough to withstand negative pressure, such that the one or more nitric oxide generating layers is not collapsed excessively and thereby may transmit negative pressure sufficiently to the wound when negative pressure is supplied to the wound dressing 100. The one or more nitric oxide generating layers may be constructed to include sufficient number or size of pores to enable transmission of negative pressure. The one or more nitric oxide generating layer may include an aperture or hole, for example, under the port, to transmit negative pressure and/or wound fluid. Further, the one or more nitric oxide generating layers may have suitable thickness(es) to transmit suitable negative pressure to the wound. For example, the one or more nitric oxide generating layers may have a thickness of about 1 mm to about 10 mm, or about 1 mm to about 7 mm, or about 1.5 mm to about 7 mm, or about 1.5 mm to about 4 mm, or about 2 mm to about 3 mm. In some embodiments, the one or more nitric oxide generating layers may have a thickness of approximately 2 mm.

In some embodiments, the layer 221 of absorbent material is provided above the transmission layer 226. The absorbent material, which can comprise a foam or non-woven natural or synthetic material, and which may optionally comprise a super-absorbent material, forms a reservoir for fluid, particularly liquid, removed from the wound site. In some embodiments, the layer 221 may also aid in drawing fluids towards the backing layer 220.

The material of the absorbent layer 221 may also prevent liquid collected in the wound dressing 100 from flowing freely within the dressing, and preferably acts so as to contain any liquid collected within the dressing. The absorbent layer 221 also helps distribute fluid throughout the layer via a wicking action so that fluid is drawn from the wound site and stored throughout the absorbent layer. This helps prevent agglomeration in areas of the absorbent layer. The capacity of the absorbent material must be sufficient to manage the exudates flow rate of a wound when negative pressure is applied. Since in use the absorbent layer experiences negative pressures the material of the absorbent layer is chosen to absorb liquid under such circumstances. A number of materials exist that are able to absorb liquid when under negative pressure, for example superabsorber material. The absorbent layer 221 may typically be manufactured from ALLEVYN™ foam, Freudenberg 114-224-4 or Chem-Posite™M11C-450. In some embodiments, the absorbent layer 221 may comprise a composite comprising superabsorbent powder, fibrous material such as cellulose, and bonding fibers. In a preferred embodiment, the composite is an air-laid, thermally-bonded composite.

In some embodiments, the absorbent layer 221 is a layer of non-woven cellulose fibers having super-absorbent material in the form of dry particles dispersed throughout. Use of the cellulose fibers introduces fast wicking elements which help quickly and evenly distribute liquid taken up by the dressing. The juxtaposition of multiple strand-like fibers leads to strong capillary action in the fibrous pad which helps distribute liquid. In this way, the super-absorbent material is efficiently supplied with liquid. The wicking action also assists in bringing liquid into contact with the upper cover layer to aid increase transpiration rates of the dressing.

An aperture, hole, or orifice 227 is preferably provided in the backing layer 220 to allow a negative pressure to be applied to the dressing 100. The fluidic connector 110 is preferably attached or sealed to the top of the backing layer 220 over the orifice 227 made into the dressing 100, and communicates negative pressure through the orifice 227. A length of tubing may be coupled at a first end to the fluidic connector 110 and at a second end to a pump unit (not shown) to allow fluids to be pumped out of the dressing. Where the fluidic connector is adhered to the top layer of the wound dressing, a length of tubing may be coupled at a first end of the fluidic connector such that the tubing, or conduit, extends away from the fluidic connector parallel or substantially parallel to the top surface of the dressing. The fluidic connector 110 may be adhered and sealed to the backing layer 220 using an adhesive such as an acrylic, cyanoacrylate, epoxy, UV curable or hot melt adhesive. The fluidic connector 110 may be formed from a soft polymer, for example a polyethylene, a polyvinyl chloride, a silicone or polyurethane having a hardness of 30 to 90 on the Shore A scale. In some embodiments, the fluidic connector 110 may be made from a soft or conformable material.

Optionally, the absorbent layer 221 includes at least one through hole 228 located so as to underlie the fluidic connector 110. The through hole 228 may in some embodiments be the same size as the opening 227 in the backing layer, or may be bigger or smaller. As illustrated in FIG. 2C a single through hole can be used to produce an opening underlying the fluidic connector 110. It will be appreciated that multiple openings could alternatively be utilized. Additionally, should more than one port be utilized according to certain embodiments of the present disclosure one or multiple openings may be made in the absorbent layer in registration with each respective fluidic connector. Although not essential to certain embodiments of the present disclosure the use of through holes in the super-absorbent layer may provide a fluid flow pathway which remains unblocked in particular when the absorbent layer is near saturation.

The aperture or through-hole 228 is preferably provided in the absorbent layer 221 beneath the orifice 227 such that the orifice is connected directly to the transmission layer 226 as illustrated in FIG. 2C. This allows the negative pressure applied to the fluidic connector 110 to be communicated to the transmission layer 226 without passing through the absorbent layer 221. This ensures that the negative pressure applied to the wound site is not inhibited by the absorbent layer as it absorbs wound exudates. In other embodiments, no aperture may be provided in the absorbent layer 221, or alternatively a plurality of apertures underlying the orifice 227 may be provided. In further alternative embodiments, additional layers such as another transmission layer or an obscuring layer such as described with in International Patent Publication WO2014/020440, the entirety of which is hereby incorporated by reference, may be provided over the absorbent layer 221 and beneath the backing layer 220.

The backing layer 220 is preferably gas impermeable, but moisture vapor permeable, and can extend across the width of the wound dressing 100. The backing layer 220, which may for example be a polyurethane film (for example, Elastollan SP9109) having a pressure sensitive adhesive on one side, is impermeable to gas and this layer thus operates to cover the wound and to seal a wound cavity over which the wound dressing is placed. In this way, an effective chamber is made between the backing layer 220 and a wound site where a negative pressure can be established. The backing layer 220 is preferably sealed to the wound contact layer 222 in a border region around the circumference of the dressing, ensuring that no air is drawn in through the border area, for example via adhesive or welding techniques. The backing layer 220 protects the wound from external bacterial contamination (bacterial barrier) and allows liquid from wound exudates to be transferred through the layer and evaporated from the film outer surface. The backing layer 220 preferably comprises two layers; a polyurethane film and an adhesive pattern spread onto the film. The polyurethane film is preferably moisture vapor permeable and may be manufactured from a material that has an increased water transmission rate when wet. In some embodiments, the moisture vapor permeability of the backing layer increases when the backing layer becomes wet. The moisture vapor permeability of the wet backing layer may be up to about ten times more than the moisture vapor permeability of the dry backing layer.

The absorbent layer 221 may be of a greater area than the transmission layer 226, such that the absorbent layer overlaps the edges of the transmission layer 226, thereby ensuring that the transmission layer does not contact the backing layer 220. This provides an outer channel of the absorbent layer 221 that is in direct contact with the wound contact layer 222, which aids more rapid absorption of exudates to the absorbent layer. Furthermore, this outer channel ensures that no liquid is able to pool around the circumference of the wound cavity, which may otherwise seep through the seal around the perimeter of the dressing leading to the formation of leaks. As illustrated in FIG. 2C, the absorbent layer 221 may define a smaller perimeter than that of the backing layer 220, such that a boundary or border region is defined between the edge of the absorbent layer 221 and the edge of the backing layer 220.

As shown in FIG. 2C, one embodiment of the wound dressing 100 comprises an aperture 228 in the absorbent layer 221 situated underneath the fluidic connector 110. In use, for example when negative pressure is applied to the dressing 100, a wound facing portion of the fluidic connector may thus come into contact with the transmission layer 226, which can thus aid in transmitting negative pressure to the wound site even when the absorbent layer 221 is filled with wound fluids. Some embodiments may have the backing layer 220 be at least partly adhered to the transmission layer 226. In some embodiments, the aperture 228 is at least 1-2 mm larger than the diameter of the wound facing portion of the fluidic connector 11, or the orifice 227.

In particular for embodiments with a single fluidic connector 110 and through hole, it may be preferable for the fluidic connector 110 and through hole to be located in an off-center position as illustrated in FIG. 2B. Such a location may permit the dressing 100 to be positioned onto a patient such that the fluidic connector 110 is raised in relation to the remainder of the dressing 100. So positioned, the fluidic connector 110 and the filter 214 may be less likely to come into contact with wound fluids that could prematurely occlude the filter 214 so as to impair the transmission of negative pressure to the wound site.

Similar to the embodiments of wound dressings described above, some wound dressings comprise a perforated wound contact layer with silicone adhesive on the skin-contact face and acrylic adhesive on the reverse. In some embodiments, the wound contact layer may be constructed from polyurethane, polyethylene or polyester. Above this bordered layer sits a transmission layer. Above the transmission layer, sits an absorbent layer. The absorbent layer can include a superabsorbent non-woven (NW) pad. The absorbent layer can over-border the transmission layer by approximately 5 mm at the perimeter. The absorbent layer can have an aperture or through-hole toward one end. The aperture can be about 10 mm in diameter. Over the transmission layer and absorbent layer lies a backing layer. The backing layer can be a high moisture vapor transmission rate (MVTR) film, pattern coated with acrylic adhesive. The high MVTR film and wound contact layer encapsulate the transmission layer and absorbent layer, creating a perimeter border of approximately 20 mm. The backing layer can have a 10 mm aperture that overlies the aperture in the absorbent layer. Above the hole can be bonded a fluidic connector that comprises a liquid-impermeable, gas-permeable semi-permeable membrane (SPM) or filter that overlies the aforementioned apertures.

FIG. 2D depicts an embodiment of a wound dressing, similar to the wound dressings of FIGS. 2A-2C. With reference to FIG. 2D, a masking or obscuring layer 2107 can be positioned beneath at least a portion of the backing layer 2140. In some embodiments, the obscuring layer 2107 can have any of the same features, materials, or other details of any of the other embodiments of the obscuring layers disclosed herein, including but not limited to having any viewing windows or holes. Examples of wound dressings with obscuring layers and viewing windows are described in International Patent Publication WO2014/020440, the entirety of which is incorporated by reference in its entirety. Additionally, the obscuring layer 2107 can be positioned adjacent to the backing layer, or can be positioned adjacent to any other dressing layer desired. In some embodiments, the obscuring layer 2107 can be adhered to or integrally formed with the backing layer. Preferably, the obscuring layer 2107 is configured to have approximately the same size and shape as the absorbent layer 2110 so as to overlay it. As such, in these embodiments the obscuring layer 2107 will be of a smaller area than the backing layer 2140.

Preferably the absorbent layer 2110 and the obscuring layer 2107 include at least one through hole 2145 located so as to underlie the port 2150. Of course, the respective holes through these various layers 2107, 2140, and 2110 may be of different sizes with respect to each other. As illustrated in FIG. 2D a single through hole can be used to produce an opening underlying the port 2150. In certain embodiments, the port may be replaced with or used in combination with a fluidic connector such as depicted in FIG. 2C. It will be appreciated that multiple openings could alternatively be utilized. Additionally, should more than one port be utilized according to certain embodiments of the present disclosure one or multiple openings may be made in the absorbent layer and the obscuring layer in registration with each respective port. Although not essential to certain embodiments of the present disclosure the use of through holes in the super-absorbent layer may provide a fluid flow pathway which remains unblocked in particular when the absorbent layer 2110 is near saturation.

The aperture or through-hole 2144 may be provided in the absorbent layer 2110 and the obscuring layer 2107 beneath the orifice 2144 such that the orifice is connected directly to the transmission layer 2105. This allows the negative pressure applied to the port 2150 to be communicated to the transmission layer 2105 without passing through the absorbent layer 2110. This ensures that the negative pressure applied to the wound site is not inhibited by the absorbent layer as it absorbs wound exudates. In other embodiments, no aperture may be provided in the absorbent layer 2110 and/or the obscuring layer 2107, or alternatively a plurality of apertures underlying the orifice 2144 may be provided.

In some embodiments, the obscuring layer 2107 can help to reduce the unsightly appearance of a dressing during use, by using materials that impart partial obscuring or masking of the dressing surface. The obscuring layer 2107 in one embodiment only partially obscures the dressing, to allow clinicians to access the information they require by observing the spread of exudate across the dressing surface. The partial masking nature of this embodiment of the obscuring layer enables a skilled clinician to perceive a different color caused by exudate, blood, by-products etc. in the dressing allowing for a visual assessment and monitoring of the extent of spread across the dressing. However, since the change in color of the dressing from its clean state to a state containing exudate is only a slight change, the patient is unlikely to notice any aesthetic difference. Reducing or eliminating a visual indicator of wound exudate from a patient's wound is likely to have a positive effect on their health, reducing stress for example.

In some embodiments, the obscuring layer can be formed from a non-woven fabric (for example, polypropylene), and may be thermally bonded using a diamond pattern with 19% bond area. In various embodiments, the obscuring layer can be hydrophobic or hydrophilic. Depending on the application, in some embodiments, a hydrophilic obscuring layer may provide added moisture vapor permeability. In some embodiments, however, hydrophobic obscuring layers may still provide sufficient moisture vapor permeability (i.e., through appropriate material selection, thickness of the obscuring layer), while also permitting better retention of dye or color in the obscuring layer. As such, dye or color may be trapped beneath the obscuring layer. In some embodiments, this may permit the obscuring layer to be colored in lighter colors or in white. In the preferred embodiment, the obscuring layer is hydrophobic. In some embodiments, the obscuring layer material can be sterilizable using ethylene oxide. Other embodiments may be sterilized using gamma irradiation, an electron beam, steam or other alternative sterilization methods. Additionally, in various embodiments the obscuring layer can be colored or pigmented, e.g., in medical blue. The obscuring layer may also be constructed from multiple layers, including a colored layer laminated or fused to a stronger uncolored layer. Preferably, the obscuring layer is odorless and exhibits minimal shedding of fibers.

### Multi-Layered Dressing for Use Without Negative Pressure

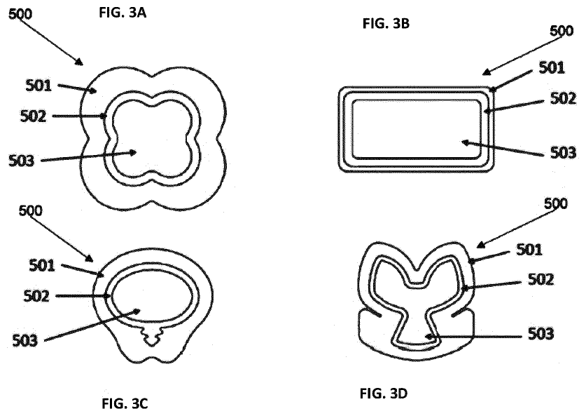

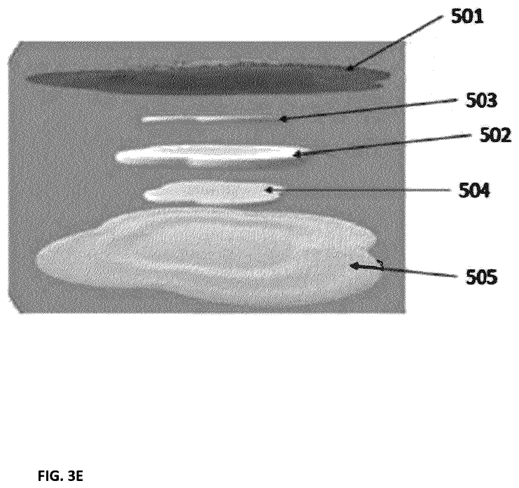

FIGS. 3A-3D illustrates various embodiments of a wound dressing 500 that can be used for healing a wound without negative pressure. FIG. 3E illustrates a cross-section of the wound dressing in FIGS. 3A-3D. As shown in the dressings of FIGS. 3A-3E, the wound dressings can have multiple layers similar to the dressings described with reference to FIGS. 2A-2D except the dressings of FIGS. 3A-3E do not include a port or fluidic connector. The wound dressings of FIGS. 3A-3E can include a cover layer 501 and an optional wound contact layer 505 as described herein. In some embodiments, the cover layer 501 may be permeable to moisture and/or air. The wound dressing can include various layers positioned between the wound contact layer 505 and cover layer 501. For example, the dressing can include one or more absorbent layers or one or more transmission layers as described herein with reference to FIGS. 2A-2D.

As shown in FIGS. 3A-3E, the dressing 500 may include a perforated wound contact layer 505 and a top film 501. Further components of the wound dressing 500 include a foam layer 504, such as a layer of polyurethane hydrocellular foam, of a suitable size to cover the recommended dimension of wounds corresponding to the particular dressing size chosen. An optional layer of activated charcoal cloth (not shown) of similar or slightly smaller dimensions than layer 504 may be provided to allow for odour control. An absorbent layer 502, such as a layer of superabsorbent air-laid material containing cellulose fibres and superabsorbent polyacrylate particulates, is provided over layer 504, of dimensions slightly larger than layer 504, and allows for an overlap of superabsorbent material and acts as leak prevention. A masking or obscuring layer 503, such as a layer of three-dimensional knitted spacer fabric, is provided over layer 502, providing protection from pressure, while allowing partial masking of the top surface of the superabsorber where coloured exudate would remain. In this embodiment this is of smaller dimension (in plan view) than the layer 502, to allow for visibility of the edge of the absorbent layer, which can be used by clinicians to assess whether the dressing needs to be changed.

The wound dressing 500 may incorporate or comprise one or more nitric oxide generating layers (e.g. a nitrite delivery layer, an acidic-group providing layer) as described herein this section or elsewhere. One of skill in the art will understand that the wound dressing 500 may incorporate any of the one or more nitric oxide generating layers disclosed herein this section or elsewhere in the specification. One of skill in the art will also understand that the one or more nitric oxide generating layers may be incorporated as a whole component layer or a part of a component layer. In some embodiments, the nitric oxide generating layers may be provided below the cover layer 501. In some embodiments, the one or more nitric oxide generating layers may be provided above the wound contact layer 505. In certain embodiments, the dressing 500 may not include the wound contact layer 505, such that one of the nitric oxide generating layers may be the lowermost layer and be configured to touch the wound surface. In some embodiments, the one or more nitric oxide generating layers may be provided below the foam layer 504. In some embodiments, the one or more nitric oxide generating layers may replace the foam layer 504. In some embodiments, the dressing 500 may include only the cover layer 501 and the one or more nitric oxide generating layers. In some embodiments, the one or more nitric oxide generating layers or components thereof (e.g., a nitrite providing layer as described herein) can be separate of the wound dressing 500. For example, the one or more nitric oxide generating layers or components thereof can be provided as a separate layer that can be placed on a wound, and the wound dressing 500 can be placed thereupon.

As described previously herein, the one or more nitric oxide generating layers, may be incorporated into or used with commercially available dressings, such as ALLEVYN™ foam, ALLEVYN™ Life, ALLEVYN™ Adhesive, ALLEVYN™ Gentle Border, ALLEVYN™ Gentle, ALLEVYN™ Ag Gentle Border, ALLEVYN™ Ag Gentle, Opsite Post-Op Visible. In some embodiments, the wound dressing 500 may include the cover layer 501, the wound contact layer 505 and the nitric oxide generating layers sandwiched therebetween. In some embodiments, the wound dressing 500 may include the cover layer 501, the absorbent layer 502, the nitric oxide generating layers below the absorbent layer 502, and the wound contact layer 505.

Further details regarding wound dressings that may be combined with or be used in addition to the embodiments described herein, are found in U.S. Pat. No. 9,877,872, issued on Jan. 30, 2018, titled “WOUND DRESSING AND METHOD OF TREATMENT,” the disclosure of which are hereby incorporated by reference in its entirety, including further details relating to embodiments of wound dressings, the wound dressing components and principles, and the materials used for the wound dressings.

Multilayered Wound Dressing with an Integrated Source of Negative Pressure

In some embodiments, a source of negative pressure (such as a pump) and some or all other components of the TNP system, such as power source(s), sensor(s), connector(s), user interface component(s) (such as button(s), switch(es), speaker(s), screen(s), etc.) and the like, can be integral with the wound dressing, such as the dressings described above in relation to FIGS. 1-3E. Additionally, some embodiments related to wound treatment comprising a wound dressing described herein may also be used in combination or in addition to those described in International Application WO 2016/174048 and International Patent Application PCT/EP2017/055225, filed on Mar. 6, 2017, entitled “WOUND TREATMENT APPARATUSES AND METHODS WITH NEGATIVE PRESSURE SOURCE INTEGRATED INTO THE WOUND DRESSING,” the disclosure of which is hereby incorporated by reference in its entirety herein, including further details relating to embodiments of wound dressings, the wound dressing components and principles, and the materials used for the wound dressings and wound dressing components.

In some embodiments, the pump and/or other electronic components can be configured to be positioned adjacent to or next to the absorbent and/or transmission layers in the wound dressing so that the pump and/or other electronic components are still part of a single apparatus to be applied to a patient with the pump and/or other electronics positioned away from the wound site.

### Nitric Oxide Generating Layers

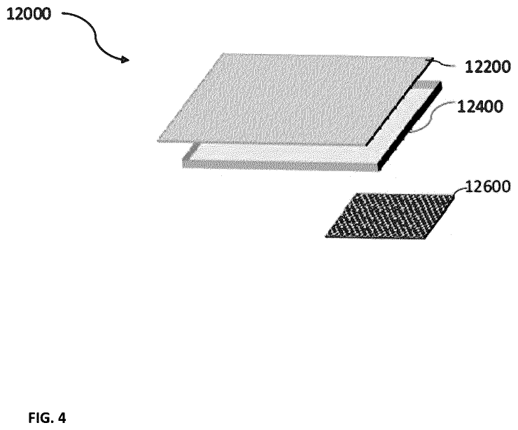

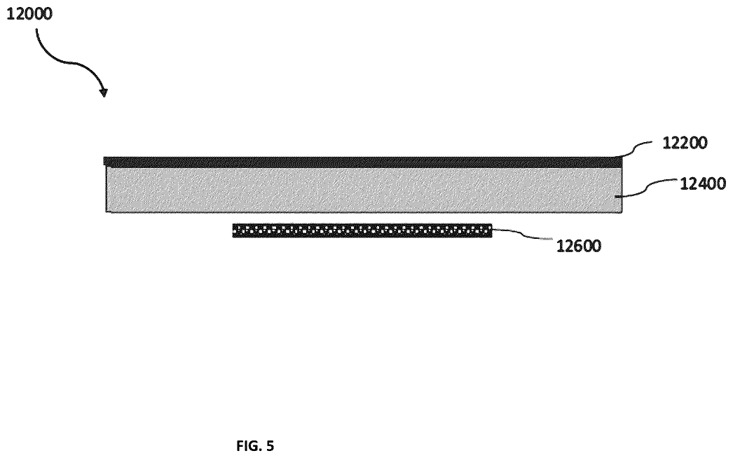

FIGS. 4-5 illustrate a wound dressing 12000 including nitric oxide generating layers according to some embodiments. In the illustrated embodiments, the wound dressing 12000 may include a cover layer 12200, an activator layer 12400, and a nitrite providing layer 12600. In some embodiments, the wound dressing 12000 may include additional layers, as further described herein. One of skill in the art will understand that although the various sections of the dressing may be referred to as “layers,” such sections may be in other suitable shapes or configurations. As will be understood by one of skill in the art, the wound dressing and/or nitric oxide delivering embodiments described herein this section or elsewhere in the specification may be applied over a wound and/or over the surrounding skin, such as the periwound area.

The cover layer 12200 may be gas impermeable, but moisture vapor permeable, and can extend across the width of the wound dressing 12000. The cover layer 12200, which may for example be a polyurethane film (for example, Elastollan SP9109 or Elastollan SP806) having a pressure sensitive adhesive on one side, may be impermeable to gas and this layer may thus operate to cover the wound and to seal a wound cavity over which the wound dressing is placed. Therefore, a chamber or a sealed wound space is made between the cover layer 12200 and the wound site. In some embodiments, negative pressure can be established within the chamber or the sealed wound space made between the cover layer 12200 and the wound site. The cover layer 12200 protects the wound from external bacterial contamination (bacterial barrier) and allows liquid from wound exudates to be transferred through the layer and evaporated from the film outer surface. The cover layer 12200 may include two or more layers, for example, a polyurethane film and an adhesive pattern spread onto the film. In certain examples, the polyurethane film may be moisture vapor permeable and may be manufactured from a material that has an increased water transmission rate when wet. In some embodiments, the moisture vapor permeability of the cover layer increases when the cover layer becomes wet. The moisture vapor permeability of the wet cover layer may be up to about ten times more than the moisture vapor permeability of the dry cover layer. In some embodiments, the cover layer 12200 may be replaced or supplemented with an additional wound dressings described elsewhere herein, such that the additional wound dressings are positioned above the nitric oxide generating layers. The cover layer may also be shower proof, such that a dressing incorporating such a cover layer may be used in the shower. The cover layer may be configured such that nitric oxide does not immediately escape through the cover layer, meaning that the cover layer is nitric oxide impermeable or semi-impermeable, thereby trapping nitric oxide against the tissue such that nitric oxide can interact with the body of a user. One of skill in the art will understand that the cover layer may be made to be both vapor permeable, but nitric oxide impermeable.

The nitrite providing layer 12600 may provide one or more nitric oxide-releasing agents at the wound site. The nitric oxide-releasing agent can include any chemical entity that yields nitric oxide at the wound site when activated or otherwise stimulated to do so. In some embodiments, the nitric oxide-releasing agent can include nitrite ion, a nitrite salt, organic and inorganic nitrites, or any pharmacologically acceptable source of nitrite such that the nitrite ion can be reduced to produce nitric oxide at the wound site. For example, the nitrite providing layer 12600 and/or element may include one or more of ammonium nitrite, lithium nitrite, calcium nitrite, sodium nitrite, potassium nitrite. In some embodiments, the nitrite providing layer may be a suitable material layer or element that includes alkali metal nitrites and/or alkaline earth metal nitrites. In certain embodiments, the nitrites may include: LiNO₂, NaNO₂, KNO₂, RbNO₂, CsNO₂, FrNO₂, Be(NO₂)₂, Mg(NO₂)₂, Ca(NO₂)₂, Sr(NO₂)₂, Ba(NO₂)₂, Ra(NO₂)₂ or any other suitable nitrite. In some embodiments, a precursor of nitrite ions, such as nitrous acid, nitrate ions, nitroprusside ions, or any pharmacologically acceptable salts thereof may be used as the source of the nitrite. In some embodiments, the nitric oxide-releasing agents may include nitrites such as nitro-functionalized compounds. For example, the nitric oxide-releasing agents may include nitroglycerine, isoamyl nitrite, isorbide mononitrate, N-(Ethoxycarbonyl)-3-(4-morpholinyl)sydnoneimine; 3-morpholinosydnonimine; 1,2,3,4-Oxatriazolium; 5-amino-3-(3,4-di-chlorophenyl)-chloride; 1,2,3,4-Oxatriazolium; 5-amino-3-(chloro-2-methyl-phenyl) chloride; 1,2,3,4-Oxatriazolium, 3-(3-chloro-2-methylphenyl)-5-[[[cyanomethylamino]carbonyl]amino]-hydroxide inner salt; S-nitroso-N-acetyl-(D,L)-penicillamine; 1-[(4′,5′-Bis(carboxymethoxy)-21-nitrophenyl)methoxy]-2-oxo-3,3, diethyl-1-triazene dipotassium salt; and [1-(4′,5′-Bis(carboymethoxy)-2′-nitropheyl) methoxy]-2-oxo-3,3-diethyl-1-triazine diacetoxymethyl ester.

In some embodiments, the nitric oxide-releasing agent of the nitrite providing layer 12600 can include diazeniumdiolates, including O-alkylated diazeniumdiolate, O-derivatized diazeniumdiolate, and non-O-derivatized diaziniumdiolate. For example, the nitric oxide-releasing agent can include diethylamine/NO, V-PYRRO/NO and/or Spermine/NO. In some embodiments, the nitric oxide-releasing agent of the nitrite providing layer 12600 can include S-nitrosothiols, such as S-nitro-gluthathione, S-nitroso-N-acetylcystein, S-nitroso-acetylpenicillamine. In some embodiments, the nitric oxide-releasing agent of the nitrite providing layer 12600 may include silica, or silica nano-particles modified with nitric oxide. In some embodiments, the nitric oxide-releasing agent can be a polymer modified with nitric oxide to include nitric oxide. For example, polyethyleneimine, polypropyleneimines, polybutyleneimines, polyurethanes or polyamides can be modified with nitric oxide to form diazeniumdiolate. In some embodiments, the nitrite providing layer 12600 may be constructed from such polymers modified with nitric oxide. Further examples of the nitric oxide-releasing agents are provided in International Publication No. WO 2006/058318, and Liang et al., “Nitric oxide generating/releasing materials”, Future Science OA, 1 (1) (2015), which are herein incorporated by reference in their entireties.

In some embodiments, the nitrite providing layer 12600 may include a nitric oxide-releasing agent (e.g. sodium nitrite) in an aqueous solution. For example, the nitrite providing layer 12600 may include a material imbibed with the nitric oxide-releasing agent (e.g. sodium nitrite) solution. In some embodiments, the nitrite providing layer 12600 may include a dry nitric oxide-releasing agent (e.g. sodium nitrite) in solid form.

The nitrite providing layer 12600 may include a mesh, a foam, a gel or any other material suitable for containing the nitric oxide-releasing agent. For example, the nitrite providing layer 12600 may include a mesh imbibed with the nitric oxide-releasing agent (e.g. sodium nitrite) solution. The mesh may be knitted, woven or non-woven. The mesh may be made of a polymeric material, for example, viscose, polyamide, polyester, polypropylene or a combination thereof. In some embodiments, the nitrite providing layer 12600 may include polypropylene, polyester, polyurethane, polyvinyl chloride, polyamide, viscose, polyester, polypropylene and/or cellulose. As described herein, the nitrite providing layer 12600 may be constructed from one or more polymers modified with nitric oxide. The nitrite providing layer 12600 could also be made of a hydrogel without acidic groups to prevent reaction with nitrite ions to emit nitric oxide. In some embodiments, the nitrite providing layer 12600 may be constructed from a colored material, such that the nitrite providing layer 12600 can be visible to assist positioning of the wound dressing 12000 during application to the wound, and to reduce the risk of incomplete removal of the nitrite providing layer 12600 from the wound after treatment. The nitrite providing layer 12600 may be fully or semi-permeable to the diffusion of nitric oxide.

In some embodiments, the nitrite providing layer 12600 is the lowermost layer of the dressing 12000, such that the nitrite providing layer 12600 may contact the wound. In some embodiments, the nitrite providing layer 12600 may be positioned within and/or over the wound. The nitrite providing layer may be constructed such that the nitrite providing layer 12600 do not substantially adhere to the skin or wound, or cause damage to the wound when in contact with the wound. In some embodiments, the dressing 12000 may include one or more layers, for example a wound contact layer, beneath the nitrite providing layer 12600. In some embodiments, the wound dressing 12000 may include two or more nitrite providing layers. For example, the wound dressing 12000 may include 2, 3, 4, 5, 6, 7 or more nitrite providing layers. In some embodiments, the nitrite providing layer 12600 can be separate of the wound dressing 12000. For example, the nitrite providing layer 12600 can be provided as a separate layer that can be placed on a wound, and the wound dressing 12000 can be placed thereupon. The nitric oxide-releasing agent may be incorporated into the nitrite providing layer to provide a nitrite dose (e.g., a sodium nitrite dose), in M (Molar) of about: 0.01 to 5.0, 0.5 to 4.5, 1.0 to 3.0, 1.0 to 2.0, and/or 1.0 to 1.5. For example, the dose may be about 0.50 M, about 0.01 M, about 1.5 M, about 2 M, or about 2.5 M.

The activator layer 12400 may contain chemical agents, functional groups or moieties which can activate and/or facilitate release of nitric oxide from the nitric oxide-releasing agent. For example, protons or acidic environment promotes the reduction of nitrites to nitric oxide, and the activator layer 12400 may include acidic groups or moieties which may provide protons in aqueous environment, thereby lowering the pH at the site of application. In certain embodiments, the acidic groups or moieties are immobilized at the activator layer 12400, for example on the surface of the activator layer 12400. The acidic groups or moieties may be covalently bonded at the activator layer 12400. In some embodiments, the activator layer 12400 may include an acidic solution. The activator layer 12400 may include a mesh, a foam, a gel or any other material suitable for containing acid groups or moieties. In some embodiments, the activator layer 12400 is positioned above the nitrite providing layer 12600 or the activator layer 12400 may be positioned below the nitrite providing layer 12600. In some embodiments, the activator layer 12400 may include proton sources such as water, methanol, ethanol, propanols, butanols, pentanols, hexanols, phenols, naphtols or polyols; aqueous acidic buffers such as phosphates, succinates, carbonates, acetates, formats, propionates, butyrates, fatty acids, amino acids, or ascorbic acids; or any suitable enzymatic or catalytic compounds. In some embodiments, body fluid such as blood, lymph, bile, or wound exudate may function as the activator, and can assist the activator layer 12400. In some embodiments, the wound dressing 12000 may not include the activator layer 12400, and wound fluid or wound exudate may function as the activator. Further examples of the activators for the nitric oxide-releasing agents are provided in International Publication No. WO 2006/058318, and Liang et al., “Nitric oxide generating/releasing materials”, Future Science OA, 1 (1) (2015), which are herein incorporated by reference in their entireties.

In some embodiments, the wound dressing 12000 may include two or more nitrite providing layers and/or two or more activator layers. For example, the wound dressing 12000 may include 2, 3, 4, 5, 6, 7 or more nitrite providing layers and/or activator layers.

In some embodiments, the activator layer 12400 includes hydrogel, such that the activator layer 12400 can absorb the wound exudate. In certain examples, the activator layer 12400 may be constructed of a xerogel. The activator layer 12400 may be constructed from any suitable materials disclosed herein. The gel of the activator layer 12400 may be presented in different physical formats. For example, the activator layer 12400 may be foamed during curing. The hydrogel may be poured into a foam and then cured in the foam. In some embodiments, the activator layer 12400 may be perforated through its thickness. The perforations may be sized to allow fluid absorption and for the desired therapeutic dose of nitric oxide to be released from the wound dressing. For example, the perforations may have a diameter sized approximately between 0.1 mm and 10 mm, between 0.15 mm and 7 mm, between 0.2 mm and 5 mm, between 0.5 mm and 4 mm or between 0.7 mm and 3 mm. The perforations may have a circular shape, a square shape, a triangular shape, or any other suitable shape. The foamed construction and/or the perforations may contribute to fluid handling capabilities of the activator layer.

In some embodiments, an activator material for the activator layer may be provided as a dispensable composition, for example as a prepolymer solution or otherwise malleable form, instead of being provided as the activator layer such as the activator layer 12400, such that it can be applied over the wound and/or around the wound more freely. For example, the activator material may be provided as gel prepolymer solution, such that it can be applied closely to or around a wound having an irregular shape size by a clinician. In some embodiments, the activator material, such as the gel prepolymer solution, may be provided in and/or applied with a syringe, and the gel prepolymer solution may have a viscosity suitable to be dispensed from the syringe. The activator material can be also formulated such that it can be rapidly cured and no longer flows once applied to or around the wound. The activator material may include an evaporative solvent, such as isopropanol. The activator material can have a suitable secondary curing mechanism, such as photoinitiated acrylate functionality. In some embodiments, the activator material can be provided as a reactive two-part system. For example, a first part and a second part may be provided to be mixed to result in polymer formation immediately before dispensing. In some embodiments, the first part and the second part may be oppositely charged flowable gels, such that they can interact on mixing to provide gels that do not flow substantially. In some embodiments, the activator material may include a material such as a gel which changes in response to the change in environment. For example, the activator material may include a material such as certain pluronics, such that it can be cured once the temperature changes as being applied from the dispenser or syringe to the skin. The activator material may be applied such that it can interact with nitrite from the nitrite providing layer 12600 (which may provide nitrite) to generate nitric oxide. Once the activator material is applied and cured or does not flow otherwise, the cover layer 12200 may be applied.

Once the dressing 12000 is activated, for example by placing the activator layer 12400 in contact with the nitrite providing layer 12600, nitric oxide-releasing agents from the nitrite providing layers 12600 releases nitric oxide. For example, in some embodiments, nitrites can be reduced to nitric oxide in the presence of an acidic environment provided by the activator layer 12400 as shown below:

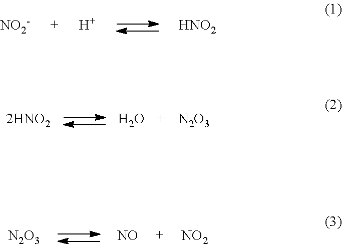

The activator layer 12400 and the nitrite providing layer 12600 may be positioned such that the nitric oxide-releasing agents can react to provide nitric oxide. For example, the activator layer 12400 and the nitrite providing layer 12600 may be in contact with each other within the dressing 12000 when in use. In some embodiments, one or more additional layers may be positioned between the activator layer 12400 and the nitrite providing layer 12600. In some embodiments, the activator layer 12400 and the nitrite providing layer 12600 may be fluidically isolated from each other before applying the dressing 12000 to the patient to prevent premature release of nitric oxide. For example, the nitrite providing layer 12600 may be provided in a packaging separate from the rest of the dressing 12000. Once the dressing 12000 is activated, the nitric oxide-releasing agents from the nitrite providing layer 12600 may disperse within the dressing 12000. In some embodiments, the nitric oxide-releasing agents may be dissolved in wound exudate and wound exudate may facilitate dispersal of the nitric oxide-releasing agents. At least a portion of the nitric oxide-releasing agents would react to release nitric oxide in the presence of the activators of the activator layer 12400. The generated nitric oxide may diffuse into the wound or be delivered to the wound by any suitable mechanisms. In some embodiments, the generated nitric oxide may not be delivered immediately or at all and is instead held within the dressing, for example by a selectively permeable membrane, such that the nitric oxide may prevent growth of or kill microbes within the dressing.

In some embodiments, the wound dressing 12000 can include a reducing agent to facilitate reduction of the nitric oxide-releasing agent (e.g. nitrite ion) to nitric oxide. Physiologically acceptable examples of such reducing agents include but are not limited to: iodide anion, ascorbic acid, ascorbate (e.g. sodium ascorbate), isoascorbates (e.g. sodium iso-ascorbate), hydroquinone, butylated quinone, tocopherol, butylated hydroquinone, hydroquinone 36ariants, butylated hydroxyanisole, butylated hydroxytoluene, beta-carotene, potassium iodide, ascorbate variants, iso-ascorbate variants, any other suitable reducing agent, and/or any of the antioxidants and/or reducing agents described herein. The reducing agent may be included in one or more layers of the wound dressing 12000. For example, the reducing agent may be included in the cover layer 12200, the activator layer 12400, the nitrite providing layer 12600, a wound contact layer (e.g., 222, 505), and/or any suitable layers of nitric oxide generating wound dressings described herein. The reducing agent may be incorporated to the one or more layers, for example, by physical entrapment, physical blending, coating, covalent bonding, or any other suitable methods. The reducing agent may be incorporated into the appropriate layer, at a w/w % of about: 0.01 to 5.0%, 0.1 to 4.5%, 1.0 to 3.0%, 1.0 to 1.5%, and/or 1.5 to 2.5%. For example, the w/w % may be about 0.02%, about 0.03%, about 0.8%, about 1.2%, about 1.4%, or about 2.43%. Higher levels of reducing agent may lead to increased production of nitric oxide; however, very high levels of reducing agent may become toxic.

As described herein, a nitric oxide source layer may include nitrite and may be referred to as a nitrite delivery layer or a nitrite providing layer in this specification. As described herein, the activator layer may include acids and may be referred to as an acid providing layer or an acid delivery layer in this specification. The nitrite providing layer/the nitrite delivery layer/the nitrite providing layer and the activator layer/the acid providing layer may be collectively or individually referred to as nitric oxide generating layer(s) in this specification.

### Nitric Oxide Dressing Materials and Construction

As will be understood by one of skill in the art, the materials and dressing constructions described above in relation to the nitric oxide delivery dressings 1200 of FIGS. 4-5 and elsewhere in the specification may include multiple suitable constructions and different types of materials. For example, the topmost layer furthest away from the wound may be a top or cover film layer, such as a top or cover layer disclosed herein, such as polyurethane materials. Such a top or cover film may be construction from materials used in the cover layer of the RENASYS drape, sold by Smith+Nephew. For example, in some embodiments the cover layer can be an IV3000 top film. Below the top or cover film layer may be a masking or fabric layer, which may be constructed of any suitable material disclosed as a masking or fabric layer herein. The masking layer may be constructed from a stretch and non-stretch polyester, polyethylene, polypropylene, polypropylethylene, and nonwovens and suitable blends constructed thereof. For example, in some embodiments the masking layer can be a 17 gsm polypropylene mask layer. Further suitable nonwovens and blend may also be utilized. In certain embodiments, the masking layer may be foam. Beneath the masking or fabric layer is an activator layer, similar to the activator layers described herein and throughout the specification. Such an activator layer may be constructed from a hydrogel adhesive, optionally containing a central polyester supporting mesh and/or supporting release liners. For example, in some embodiments the activator layer can be a DURAFIBER format loaded with a hydrogel as described herein. DURAFIBER by Smith+Nephew is a commercially available absorbent non-woven material, made from cellulose. In such an example, depending on the dressing size, an equivalent of about 6 grams of hydrogel can be loaded onto a 10.8 cm×10.8 cm piece of DURAFIBER, which can be cut down to 10 cm×10 cm resulting in about 5.14 grams of the hydrogel on the 10 cm×10 cm piece of DURAFIBER. The activator layer may be constructed from any suitable hydrogel material disclosed herein such as an acrylic acid hydrogel and/or a sulfonic acid hydrogel. Below the activator layer may be an acquisition distribution layer, which may be constructed of any suitable acquisition distribution layer materials disclosed herein, such as in relation to FIGS. 2C-2D. For example, the acquisition distribution layer may be constructed from 3-D knit, gauze and/or stretch polyester fibers woven into a net format, similar to the material used in Acticoat Flex by Smith+Nephew, although silver is optional. In some embodiments the acquisition distribution layer may be constructed from a pre-polymer solution with a mixture of water, surfactant, and polyethylene glycol such as foams used in Allevyn foam by Smith+Nephew. The masking layer and acquisition distribution layer may use the same materials and be interchangeable. In certain embodiments, the acquisition distribution layer may be pressed into the activator layer and/or cured into the activator layer. Curing the acquisition distribution layer into the activator layer may increase the rate of nitric oxide formation due to more rapid transport. Under the acquisition distribution layer, there may be a wound contact layer which may be constructed from any suitable material disclosed herein, such as in relation to FIGS. 2C-2D. For example, the wound contact layer may include a silicone adhesive and perforated polyurethane film. The wound contact layer may include an acrylic adhesive. A nitrite providing layer, constructed from any suitable materials disclosed herein, may be positioned beneath the wound contact layer such that the nitrite providing layer is directly against a wound or other tissue. In some embodiments, the nitrite providing layer may be in other positions, such as above the activator layer and/or elsewhere in the dressing. For example, in some embodiments the nitrite providing layer can be a separate 17 gsm polypropylene mesh saturated with a sodium nitrite solution. In certain embodiments, the ALLEVYN or PICO dressings disclosed in FIGS. 2-3 may be placed directly over an activator layer and underlying nitrite providing layer. Placing the nitrite providing layer directly against the wound, periwound area, and/or other tissues may allow for increased release of nitric oxide directly into the tissue. As will be understood by one of skill in the art, the wound dressing and/or nitric oxide delivering embodiments described herein this section or elsewhere in the specification may be applied over a wound and/or over the surrounding skin, such as the periwound area.

### Chemiluminescence

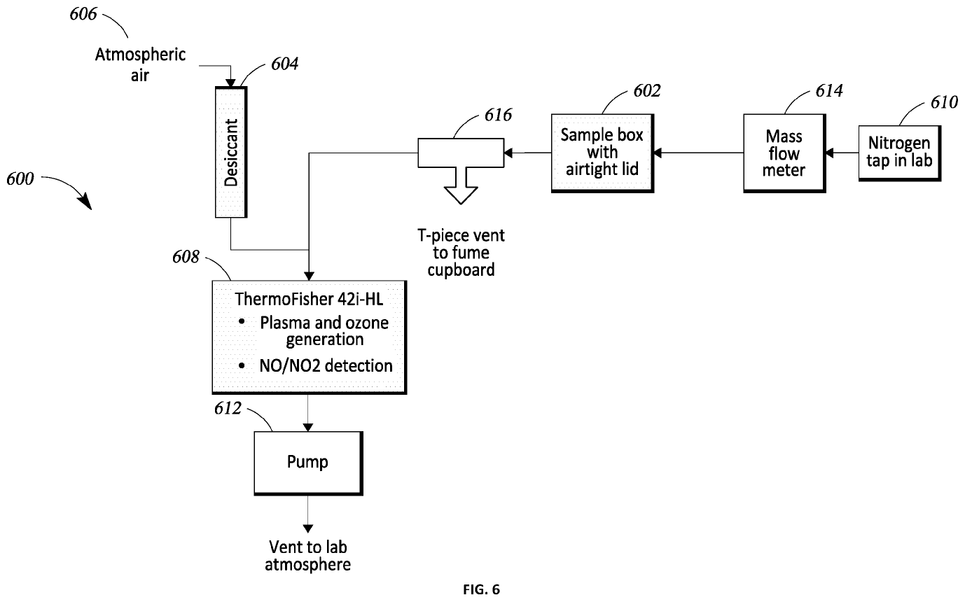

FIG. 6 shows an example setup 600 for a chemiluminescence protocol for testing a nitric oxide delivery dressing such as disclosed above in relation to FIGS. 4 and 5. The protocol may include a sample box 602, desiccant 604, an atmospheric air source 606, a chemiluminescence detector 608, a nitrogen supply 610, an air pump 612, a mass flow meter 614, and T-piece connector 616. In certain embodiments, a ThermoFisher 42i-HL detector may be used as a chemiluminescence detector 608. After warming up the equipment with air flow under atmospheric pressure, the sample box 602 and nitrogen supply can be connected to the equipment. The nitrogen flow through the mass flow controller may be set to a suitable value, such as between about: 1 to 100, 10 to 90, 25 to 75, 40 to 60, or about 50 mL/min. After flushing the system (such as for about 1 to 60, 10 to 50, 20 to 40, or about 30 minutes), a nitrite providing layer (such as a nitrite mesh) and activator layer (such as an acid providing hydrogel) may be placed in the sample chamber 602. In embodiments, the nitrite mesh is smaller in total area than the activator layer. In particular embodiments, the nitrite providing layer and/or the activator layer may have a length and/or a width of about 0.5 to 20, 1 to 10, 2 to 8, or about 4 to 6 centimeters. In certain embodiments, the nitrite providing layer may be 2.5 cm×2.5 cm while the activator layer is 3 cm×3 cm.

NO/NO₂ release concentrations may be measured by the chemiluminescence detector at an appropriate rate, checking the concentrations in ppb or ppm and monitoring periodically, such as about every 1, 2, 5, 10, 30, 60 or 90 seconds. In certain embodiments, the NO/NO₂ concentration may be checked in ppm.

As will be understood by one of skill in the art, maximizing NO over NO₂ is desirable for the dressings disclosed herein, such as the dressings described in relation to FIGS. 4-5. While nitrogen dioxide (NO₂) may exert antimicrobial properties, NO₂ does not have the vasodilating properties nor the capability of activating cell proliferation of NO. It is therefore generally desirable to reduce the generation of NO₂ as far as possible in the acidification of nitrites such as by such means as reducing the oxidation of dissolved nitric oxide (NO) by removing the oxygen from the body of the hydrogel where the acidification of nitrite takes place. The nitric oxide delivery dressings disclosed herein may produce both NO and NO₂. In some embodiments, the nitric oxide dressings disclosed herein may produce NO and NO₂ in a ratio of NO/NO₂ such as about 0.5:1 to 500:1, 1:1 to 400:1, 10:1 to 300:1, 20:1 to 200:1. 50:1 to 100:1. For example, the ratio may be about or at least about 0.5:1 1.01:1, 1.1:1, 1:1, 2:1. 5:1, 10:1, 20:1, 30:1, 50:1, 100:1, 200:1, or 500:1.

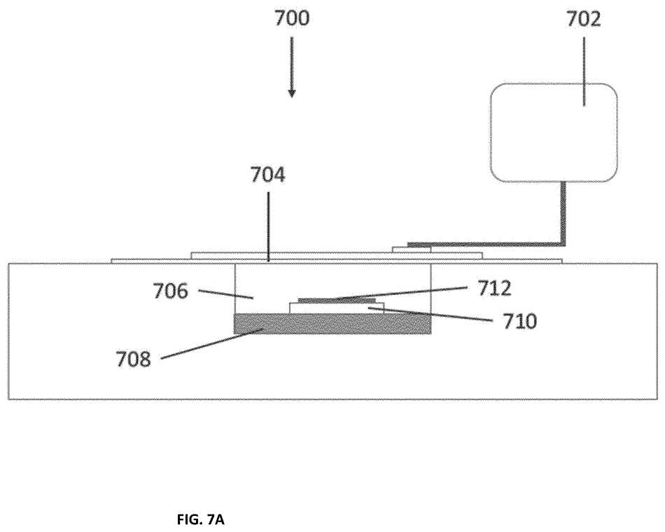

FIG. 7 shows an example of an experimental set-up 700 demonstrating nitric oxide delivery from a combination of activator layer and nitrite providing layer, similar to the dressings described in relation to FIGS. 4 and 5, while under negative pressure. As shown in FIG. 7, a negative pressure wound therapy pump 702 is connected to a negative pressure wound therapy dressing 704 such as described herein in FIGS. 2A-2D. The dressing is sealed over a chamber 706 containing nitrite test solution 708 which changes color in the presence of NO. Prior to applying negative pressure, the test solution did not change color. After running negative pressure for a period of time to ensure that no background color change occurred, an activator layer such as described herein (such as an acid-providing hydrogel), was placed in the chamber and negative pressure was applied. Again, no color change occurred. Lastly, a nitrite providing layer such as described herein (such as a sodium nitrite mesh) was placed onto the activator layer without having the nitrite providing layer touch the nitrite test solution, and negative pressure was applied. After 15 minutes of negative pressure, the indicator solution changed color, thereby demonstrating that interaction between the activator layer and the nitric oxide layer can produce nitric oxide, even while under negative pressure.

As will be understood by one of skill in the art, negative pressure may be applied to any of the nitric oxide delivering dressings disclosed herein, such as the dressings described in FIGS. 4-5 and elsewhere in the specification. A dressing, such as the dressings described in FIGS. 2A-2D may be placed over an activator layer and nitrite providing layer which are placed in a wound, thereby delivering nitric oxide to a wound while simultaneously applying negative pressure wound therapy.

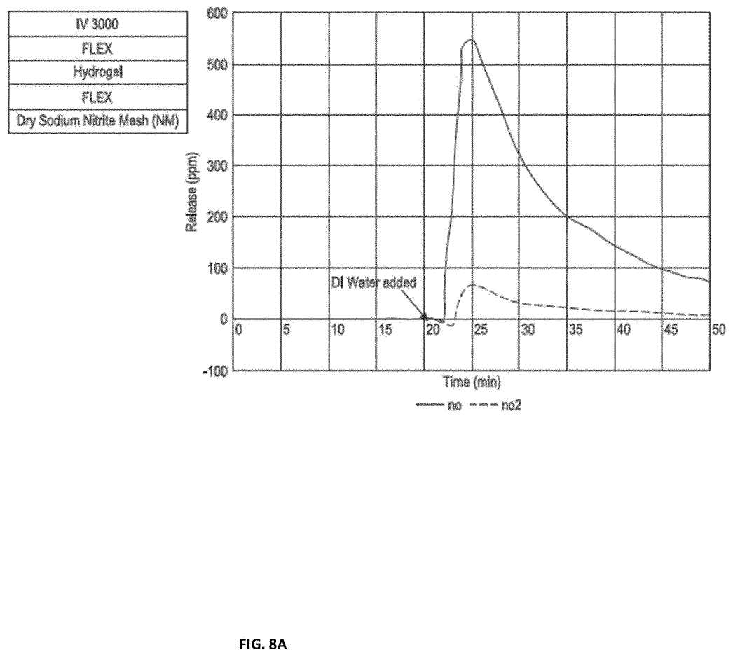

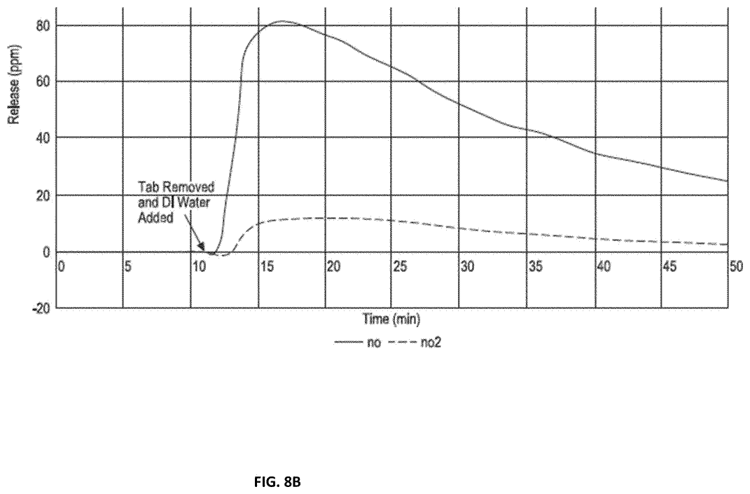

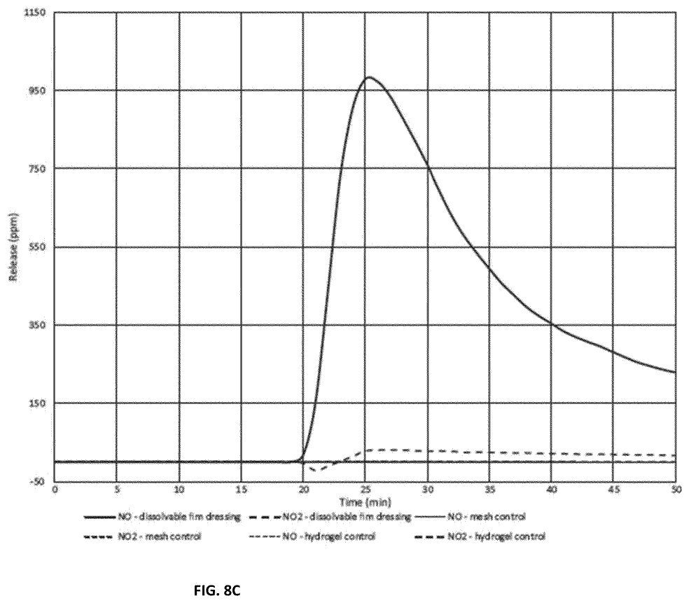

FIGS. 8A through 8C show examples of chemiluminescence experimental runs using a protocol similar to that described above. As will be understood by one of skill in the art, these measurements taken in these experimental runs are merely exemplary and the disclosures herein are not limited to such values. FIG. 8A shows the experimental results when testing a dry sodium nitrate mesh embodiment with the arrangement shown in FIG. 8A, including a polyurethane cover layer overlying a stretch polyester ADL layer, positioned over a hydrogel activator layer sandwiched between another stretch polyester ADL layer over a dry sodium nitrite mesh as shown in the figure. In this experimental run, after the DI water was added, the dry sodium nitrate mesh released approximately 550 ppm NO and 75 ppm NO₂ at its peak at the 25 minute mark, slowly reducing in concentration to approximately 80 ppm NO and 10 ppm NO₂ at the 50 minute mark.

FIG. 8B shows the experimental results when testing a full dressing design with a pull-out tab and self-sealing borders. The pull out tab is used to initially separate the nitrite providing layer from the activator layer, therefore when the tab is removed and the dressing becomes wet, the interaction between the nitrite providing layer and the activator layer produces nitric oxide. In this experimental run, after the DI water was added, the full dressing design with the pull-out tab and self-sealing borders released approximately 84 ppm NO and 15 ppm NO₂ at its peak at the 17 minute mark, slowly reducing in concentration to approximately 25 ppm NO and 5 ppm NO₂ at the 50 minute mark.

FIG. 8C shows an example of the experimental results for a dressing containing a degradable film. Here, a degradable film was placed between the activator layer and the nitrite providing layer, thereby generating nitric oxide once the degradable layer breaks down. In this experimental run, after the DI water was added, the dressing containing a degradable film released approximately 1000 ppm NO and 45 ppm NO₂ at its peak at the 25 minute mark, slowly reducing in concentration to approximately 225 ppm NO and 20 ppm NO₂ at the 50 minute mark. The experimental protocol was also utilized to test an activator layer containing sodium iso-ascorbate. In this experimental run, after the DI water was added, the activator layer containing sodium iso-ascorbate released approximately 52 ppm NO and 4 ppm NO₂ at its first peak at the 80 minute mark, 66 ppm NO and 5 ppm NO₂ at its second and maximum peak at the 110 minute mark slowly reducing in concentration to approximately 45 ppm NO and 2 ppm NO₂ at the 160 minute mark.

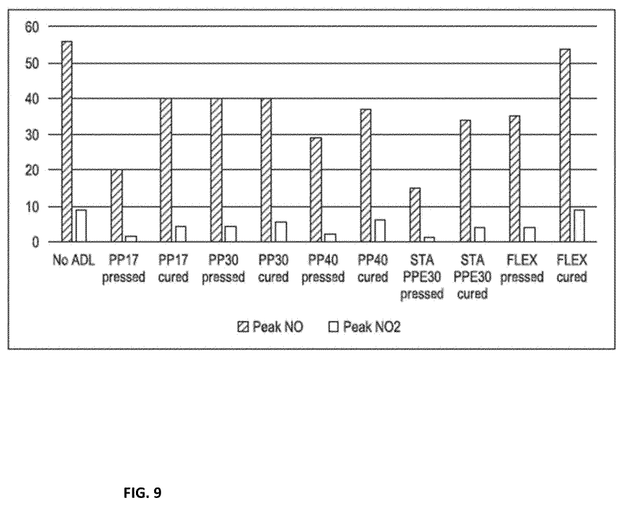

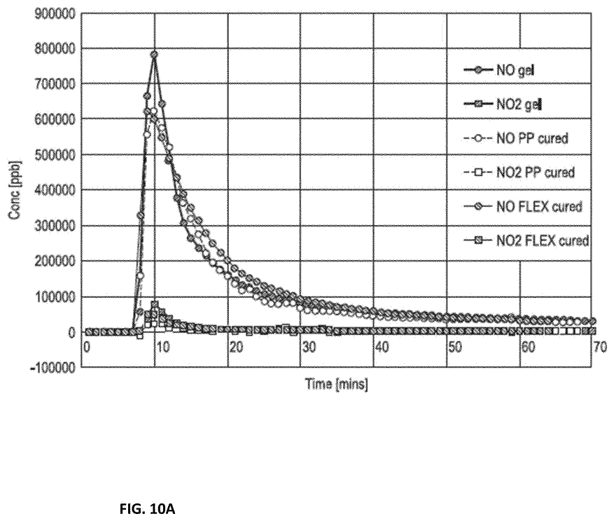

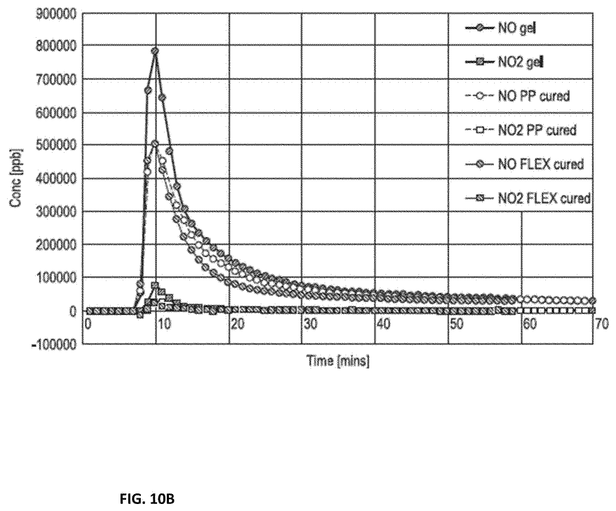

FIG. 9 shows an example of the relative peak output in ppm for activator hydrogels (acid providing) either with an acquisition distribution layer or without, including polypropylene, polypropylethylene, or stretch polyester acquisition distribution layers with various gsm (g/m²). With no acquisition distribution layer, the peak NO and NO₂ concentrations were approximately 55 ppm and 10 ppm respectively; however, one of skill in the art will understand that an acquisition distribution layer may allow for improved fluid distribution and handling throughout a larger area such as a dressing. With a 17 gsm polypropylene pressed acquisition distribution layer, the peak NO and NO₂ concentrations were approximately 20 ppm and 2 ppm respectively. With a 17 gsm polypropylene cured acquisition distribution layer, the peak NO and NO₂ concentrations were approximately 40 ppm and 5 ppm respectively. As explained above, curing the acquisition distribution layer may allow for increased fluid transport and an increased rate of nitric oxide formation. With a polypropylene 30 g/m² pressed acquisition distribution layer, the peak NO and NO₂ concentrations were approximately 40 ppm and 5 ppm respectively. With a polypropylene 30 g/m² acquisition distribution layer, the peak NO and NO₂ concentrations were approximately 40 ppm and 5 ppm respectively. With a polypropylene 40 g/m² pressed acquisition distribution layer, the peak NO and NO₂ concentrations were approximately 30 ppm and 2 ppm respectively. With a polypropylene 40 g/m² cured acquisition distribution layer, the peak NO and NO₂ concentrations were approximately 38 ppm and 5 ppm respectively. With a polypropylethylene 30 g/m² pressed acquisition distribution layer, the peak NO and NO₂ concentrations were approximately 35 ppm and 3 ppm respectively. With a polypropylethylene 30 g/m² cured acquisition distribution layer, the peak NO and NO₂ concentrations were approximately 35 ppm and 3 ppm respectively. With a stretch polyester pressed acquisition distribution layer, the peak NO and NO₂ concentrations were approximately 35 ppm and 3 ppm respectively. With a FLEX pressed acquisition distribution layer, the peak NO and NO₂ concentrations were approximately 55 ppm and 8 ppm respectively.

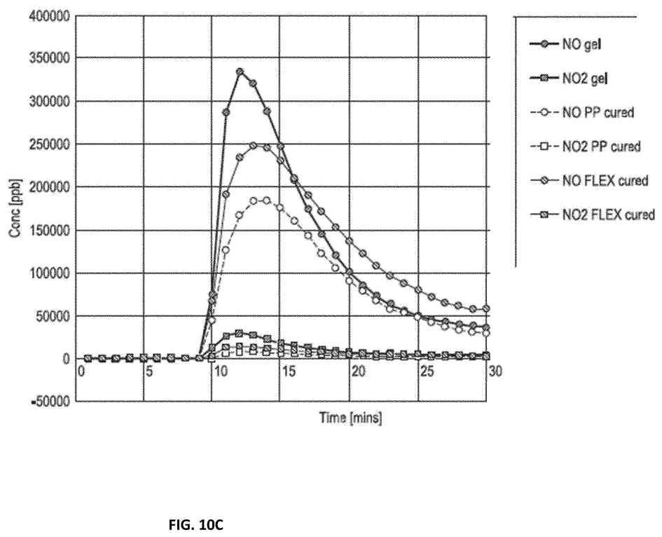

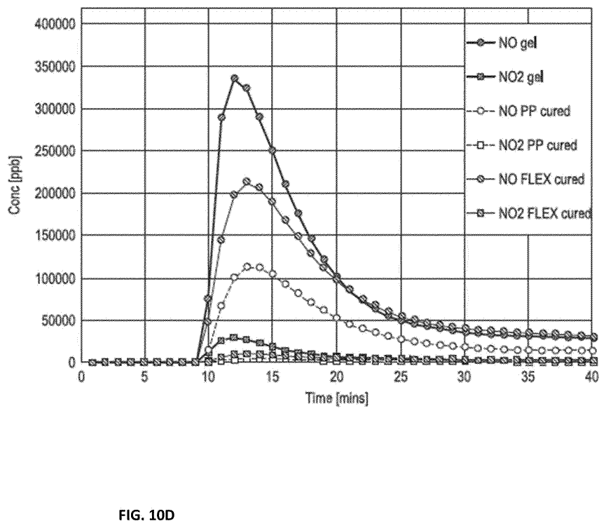

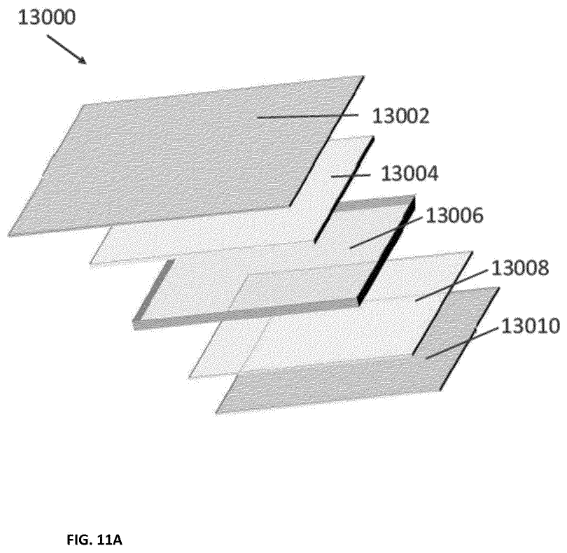

FIGS. 10A through 10D show examples of the concentration of NO and NO₂ over time for several embodiments incorporating an activator layer and nitric oxide providing layer. As shown in FIGS. 10A-10B, an activator layer containing approximately 2-3% sodium iso-ascorbate was tested with or without different acquisition distribution layers that were pressed or cured. The gel with no acquisition distribution layer produced (p indicating peak) pNO=785 ppm and pNO2=78 ppm. The activator layer with a stretch polyester pressed into the gel produced pNO=506 ppm and pNO2=24 ppm. For stretch polyester cured on the activator layer, pNO=625 ppm; pNO2=50 ppm. For polypropylene pressed into the gel, the pNO=508 ppm and pNO2=26 ppm. For polypropylene cured into the gel, the pNO=624 ppm and pNO2=26 ppm.

FIGS. 10C-10D show examples of the concentration of NO and NO₂ over time for an activator layer containing approximately 1-2% sodium iso-ascorbate with or without different acquisition distribution layers that were pressed or cured. The activator layer with no ADL produced pNO=334 ppm; pNO2=40 ppm. For the stretch polyester acquisition distribution layer pressed into the activator layer, pNO=211 ppm and pNO2=10 ppm. For the stretch polyester acquisition distribution layer cured into the activator layer, pNO=247 ppm and pNO2=14 ppm. For the polypropylene acquisition distribution layer pressed into the activator layer, pNO=112 ppm and pNO2=5 ppm. For the polypropylene acquisition distribution layer cured into the activator layer, pNO=184 ppm and pNO2-8 ppm. As explained elsewhere in the specification, curing an acquisition distribution layer into an activator layer may improve fluid handling and nitric oxide production relative to nitrogen dioxide production.

### Xerogels and Hydrogel Construction

Reference may be made throughout the specification to xerogels. A xerogel may be formed from a gel by drying with unhindered shrinkage. As will be understood by one of skill in the art, a xerogel is a gel that has very low free water content, so low that minimal reaction to form nitric oxide will occur without the addition of further water and/or liquid. For example, a xerogel may be substantially free of water in the dry state. Drying may be completed by any suitable means known in the art (e.g., freeze drying).

In certain examples, hydrogels (which may subsequently become xerogels after drying) may be generated with or without glycerol, and may contain a standard amount or double, triple, or quadruple the required amount of crosslinker PEG diacrylate as needed. A 2-Acrylamido-2-methyl-1-propanesulfonic acid sodium salt solution may be present in the xerogel. Hydrogels and xerogels may be created by converting 2-acrylamido-2-methyl-1-propanesulphonic acid (SA), 43 ariants 43 e with MEHQ as supplied, to a sodium salt by dissolving into water, then 43ariants43ed43 with 50% NaOH to pH 7.0 with cooling from a 10C water bath to form a solution of the 43ariants43ed acid (NaAMPS). Hydrogels can contain about 5.393 wt % 2-acrylamido-2-methyl-1-propanesulphonic acid (equating to 1.0 SA), about 4.654 wt % 2-acrylamido-2-methyl-1-propanesulphonic acid (equating to 0.85 SA), about 2.839 wt % 2-acrylamido-2-methyl-1-propanesulphonic acid (equating to 0.5 SA), and/or a range of between about 1.457 wt % 2-acrylamido-2-methyl-1-propanesulphonic acid (equating to 0.25 SA) to about 7.704 wt % 2-acrylamido-2-methyl-1-propanesulphonic acid (equating to 1.5 SA). Hydrogel prepolymers may be prepared by predispersing 2-hydroxy-2-methylpropiophenone photoinitiator into PEG diacrylate under minimal light, then mixing for 10-20 mins with a mixture of 58% aqueous sodium 2-acrylamido-2-methyl-1-propanesulfonate solution (Na AMPS), Sodium iso-ascorbate, pre-ground 2-Acrylamido-2-methyl-1-propanesulfonic acid (AMPS acid) and glycerol. The AMPS acid may be fully dissolved in the stirred Na AMPS solution prior to slowly adding the glycerol, and then the photoinitiator/diacrylate mixture in a water bath. In certain embodiments, hydrogels may also be prepared with twice the normal amount of photoinitiator/crosslinker and/or the omission of glycerol and/or using triple the amounts of prepolymer mix in the molds to form gels with three times the thickness.

### Nitric Oxide Generating Dressings Utilizing Sodium Nitrite

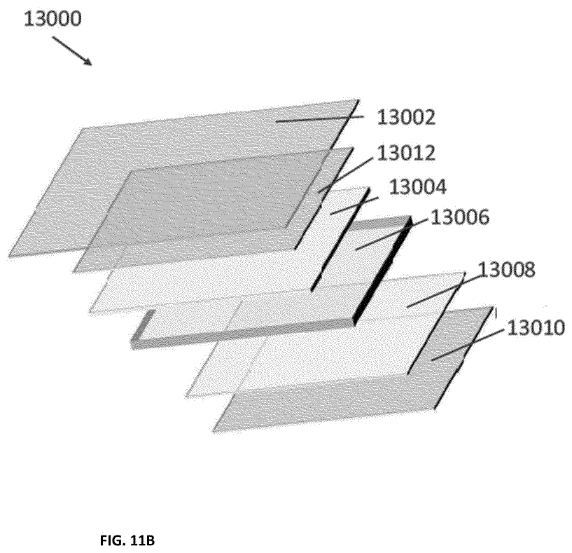

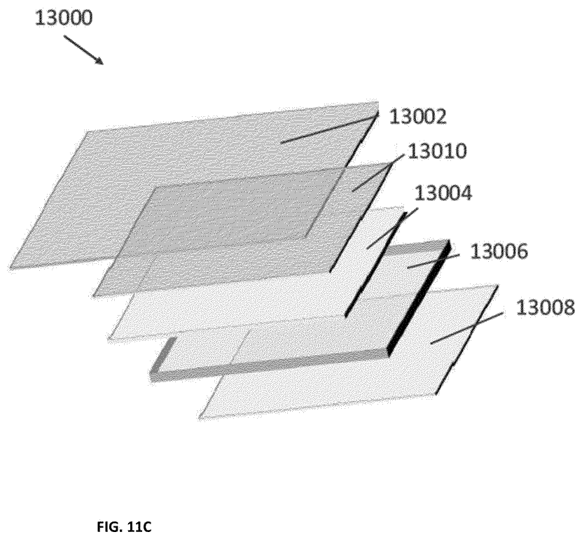

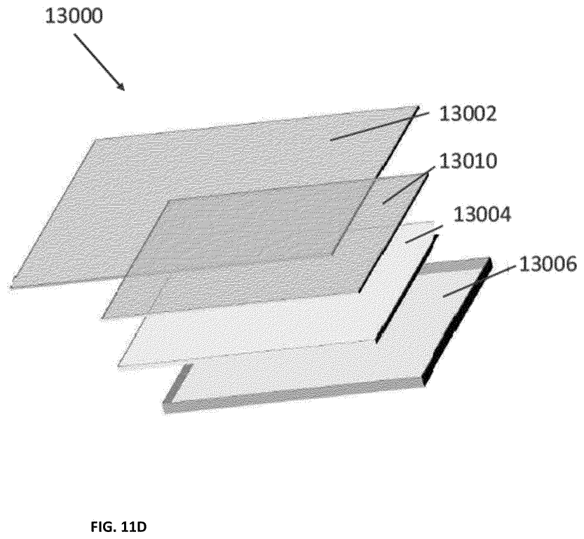

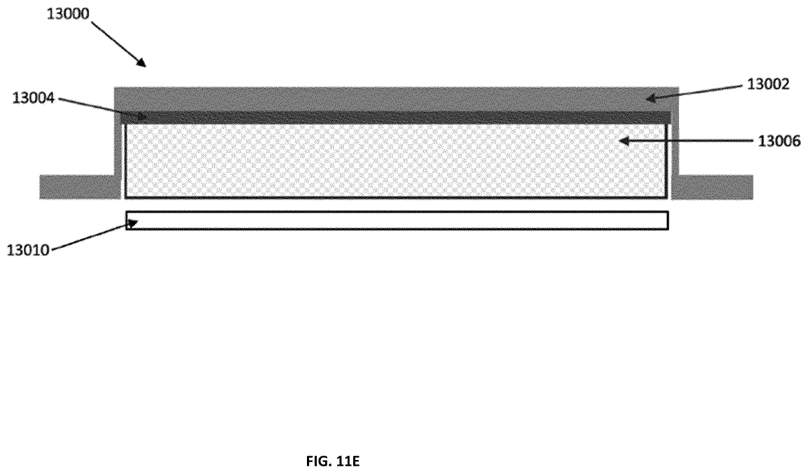

FIGS. 11A-11E depict embodiments of a nitric oxide generating wound dressing with various arrangements of layers. One of skill in the art will understand that the various layers depicted in FIGS. 11A-11E may be ordered in any suitable order and the orders depicted in the figures are merely examples. Furthermore, one of skill in the art will understand that one or more layers may be omitted, and/or the wound dressing can include multiples of one or more of the layers shown. In some embodiments, the uppermost layer may be a cover layer 13002, which may have any of the same features, materials, or other details of cover layers disclosed herein, such as being constructed from a film. Said cover layer 13002 may be suitable for sealing a dressing over a wound and for connecting to a source of negative pressure and/or for maintaining negative pressure at a wound site. In certain embodiments, the border region of the cover layer 13002 may be attached to the skin around the wound, forming a seal, such that the wound exudate can be contained within the wound dressing 13000. Below the cover layer, there may be a masking or obscuring layer 13004 (heretofore referred to as a “masking layer”) to prevent or limit visualization of the wound or the wound exudate through the cover layer 13002. The masking layer 13004 may be positioned beneath at least a portion of the cover layer 13002. In some embodiments, the masking layer 13004 can have any of the same features, materials, or other details of any of the other embodiments of the masking layers disclosed herein, including but not limited to having any viewing windows or holes. Examples of wound dressings with obscuring layers and viewing windows are described in International Patent Publications WO2013/007973 and WO2014/020440, the entireties of which are incorporated by reference. Additionally, the masking layer 13004 may be positioned adjacent to the cover layer, or can be positioned adjacent to any other dressing layer as desired. In the illustrated embodiment, the masking layer 13004 is positioned between the cover layer 13002 and the activator layer 13006. As explained elsewhere herein and as will be understood by one of skill in the art, the activator layer may be an acid providing layer or other suitable layer. In certain embodiments, the masking layer 13004 can be adhered to or integrally formed with the cover layer 13002. The masking layer 13004 may be configured to have approximately the same size and shape as the activator layer 13006 so as to overlay it. The masking layer 13004 may be of a smaller area than the cover layer 13002. In certain embodiments, the masking layer 13004 can horizontally wick fluid and may function as an acquisition distribution layer as well.

In particular embodiments, the activator layer 13006 may have any of the same features, materials, or other details of any of the other embodiments of activator layers disclosed herein. For example, the activator layer 13006 may be adhesive and may be constructed of a hydrogel or xerogel configured to have a plurality of acidic groups or moieties which may provide protons in an aqueous environment. As explained elsewhere in the specification, under such acidic conditions nitrite ions from a nitrite providing layer 13010 may be reduced to nitric oxide for delivery to a wound or intact skin. As explained elsewhere herein and as will be understood by one of skill in the art, the activator layer may be a nitrite providing layer or other suitable layer. The activator layer 13006 (e.g. hydrogel layer) may include a plurality of perforations that extend through the thickness of the activator layer, as described elsewhere herein. The plurality of perforations may allow or facilitate passage of wound exudate through the activator layer, such that wound exudate below or around the activator layer can be transported to one or more additional absorbing layers and/or an evaporative layer or layers (e.g. cover layer) above the activator layer, thus preventing excessive buildup of wound exudate below the activator layer 13006. Additionally, the plurality of perforations may provide increased surface area of the activator layer, thereby increasing the absorption rate of the activator layer.

As shown in FIG. 11A, in some embodiments, an acquisition distribution layer 13008 may be placed between the activator layer 13006 and the nitrite providing layer 13010. In certain embodiments, the acquisition distribution layer 13008 may be constructed so as to advantageously horizontally wick fluid, such as wound exudate, as it is absorbed through the layers of the dressing 13000. Such lateral wicking of fluid may allow maximum distribution of the fluid through the activator layer 13006, enabling the activator layer 13006 to reach its full holding capacity. Further, acquisition distribution layer 13008 may facilitate nitric oxide production, as nitrite ion dissolved in fluid may spread across the surface of the activator layer 13006 more quickly. Some embodiments of the acquisition distribution layer 13008 may comprise viscose, polyester, polypropylene, cellulose, or a combination of some or all of these, and the material may be needle-punched. Some embodiments of the acquisition distribution layer 13008 may comprise cellulose in the range of 40-160 gsm (or about 40 to about 160 gsm), for example 80 (or about 80) gsm. Some embodiments of the acquisition distribution layer 13008 may comprise polyethylene in the range of 40-150 grams per square meter (gsm). In some embodiments, the acquisition distribution layer 13008 may have a thickness of 1.2 mm or about 1.2 mm, or may have a thickness in the range of about 0.5 mm to about 3.0 mm, about 0.5 mm to about 3.0 mm., 0.7 mm to 2.5 mm, 0.9 mm to 2.1 mm, or 1.1 mm to 1.5 mm. In certain embodiments, the acquisition distribution layer 13008 may be constructed from a material which resists compression under the levels of negative pressure commonly applied during negative pressure therapy.

The acquisition distribution layer 13008 may include a plurality of loosely packed fibers, which may be arranged in a substantially horizontal fibrous network. In some embodiments, the acquisition distribution layer 13008 may consist of a mix of two fiber types. One may be a flat fiber which may be 20 μm to 50 μm in width, or approximately 20 μm to approximately 50 μm in width, and may comprise a cellulosic based material. The other fiber may be a two component fiber that has an inner core that is 8 μm to 10 μm in diameter, approximately 8 μm to approximately 10 μm in diameter, 7 μm to 11 μm in diameter, 6 μm to 12 μm in diameter, or 5 μm to 13 μm in diameter and an outer layer with a thickness of 1 μm to 2 μm, approximately 1 μm to approximately 2 μm, 1 μm to 2.3 μm, 0.8 μm to 2.5 μm, or 0.5 μm to 3 μm. The two component fiber may be a mix of a polyethylene (PE) type material, and polyethylene terephthalate (PET). In some embodiments the inner core of the two component fiber may be PET and the outer layer may be PE. The PE/PET fibers may have a smooth surface morphology, while the cellulosic fibers may have a relatively rougher surface morphology. In some embodiments the ADL material may comprise about 60% to about 90% cellulosic fibers, for example approximately 75% cellulosic fibers, and may comprise about 10% to about 40% PE/PET fibers, for example approximately 25% PE/PET fibers. In some embodiments, the acquisition distribution layer 13008 may include split microfibers.

A majority of the fiber volume may extend horizontally (that is, parallel to the plane of the top and bottom surfaces of the material), or substantially or generally horizontally. In another embodiment, 80%-90% (or approximately 80% to approximately 90%) or more of the fiber volume may extend horizontally, or substantially or generally horizontally. In another embodiment, all or substantially all of the fiber volume may extend horizontally, or substantially or generally horizontally. In some embodiments, a majority, 80%-90% (or approximately 80% to approximately 90%) of the fibers or more, or even all or substantially all of the fibers, span a distance perpendicular to the thickness of the acquisition distribution layer 13008 (a horizontal or lateral distance) that is greater than the thickness of the acquisition distribution layer 13008. In some embodiments, the horizontal or lateral distance spanned by such fibers is 2 times (or about 2 times) or more, 3 times (or about 3 times) or more, 4 times (or about 4 times) or more, 5 times (or about 5 times) or more, or 10 times (or about 10 times) or more the thickness of the acquisition distribution layer 13008. The orientation of such fibers may promote lateral wicking of fluid through the acquisition distribution layer 13008. This may more evenly distribute fluid such as wound exudate throughout the acquisition distribution layer 13008. In some embodiments, the ratio of the amount of fluid wicked laterally across the acquisition distribution layer 13008 to the amount of fluid wicked vertically through the acquisition distribution layer 13008 under negative pressure may be 2:1 or more, or approximately 2:1 or more, or may be up to 10:1 or more, or approximately 10:1 or more, in some embodiments.

Continuing with FIG. 11A, in embodiments a nitrite providing layer 13010 may be provided beneath the acquisition distribution layer 13008. Such a nitrite providing layer 13010 may have any of the same features, materials, or other details of any of the other embodiments of nitrite providing layers disclosed herein, for instance, the nitrite providing layer 13010 may be a nitrite providing layer. For example, the nitrite providing layer may be a wet mesh imbued with sodium nitrite solution. In some embodiments, the nitrite providing layer 13010 may be dry and include a dry nitrite source, such as dry sodium nitrite. Such dry sodium nitrite may be loaded into a material layer, said material layer constructed from a suitable material such as any material disclosed herein. As will be understood by one of skill in the art, a dry material and/or substance is one that is free or relatively free of liquid. For example, polypropylene, polyethylene, or melt extrudable fibers may be suitable materials for such a layer. In embodiments, such a nitrite providing layer 13010 layer may need to be separated initially from the activator layer 13006 when the activator layer is a hydrogel so as to avoid reaction and production of nitric oxide prior to application to a wound and/or skin. As depicted in FIG. 11A a dry fluid acquisition distribution layer 13008 may serve to separate the nitrite providing layer 13010 and a hydrogel activator layer 13006 prior to application. However, such a dry sodium nitrite providing layer may be adjacent to a xerogel activator layer 13006, since a xerogel will not be wet. In the instance of a xerogel, activation may occur upon contact with fluid such as wound exudate as the wound exudate wicks through the dressing. In the instance of a hydrogel, once fluid such as wound exudate comes into contact with the acquisition distribution layer 13008, then nitrite ions may come into contact with the acidic environment created by the activator layer, thereby generating nitric oxide which may then migrate into the wound and/or skin. In some embodiments, each of the layers such as the nitrite providing layer, the activator layer, and any other suitable layer may be stored dry prior to use. Prior to application to the skin or wound, the layers may be wet by a suitable liquid such as saline.

As depicted in FIG. 11B, to maintain nitric oxide release there could be a number of layers containing dry sodium nitrite, for example a first nitrite providing layer 13010 and a second nitrite providing layer 13012 that would be ‘activated’ as wound fluid reaches and wets out the layer(s), enabling the sodium nitrite to come into contact with the acidic groups of the hydrogel or xerogel of activator layer 13006, thereby producing nitric oxide. In certain embodiments, there may be 2, 3, 4, 5, 6 or more layers containing dry sodium nitrite. As shown in FIG. 11B, the masking layer 13004 may serve to prevent contact between the second nitrite providing layer 13012 and the activator layer 13006. In certain embodiments, additional acquisition distribution layers and/or masking layers may be sandwiched with activator layers to provide additional sources of nitric oxide.

As depicted in FIGS. 11C-11D, in some embodiments the activator layer 13006 may be positioned beneath the nitrite providing layer, thereby relying on the dressing wetting out (such as from wound exudate) and activating the nitrite providing layer 13010.

As depicted in FIG. 11E, in some embodiments the wound dressing 13000 can have a cover layer 13002 as described herein as its uppermost layer. For example, the cover layer 13002 can include an IV3000 top film and border. A masking or obscuring layer 13004 as described herein can be positioned below at least a portion of the cover layer 13002. For example, the masking layer 13004 can include a 17 gsm polypropylene mesh. An activator layer 13006 as described herein can be positioned below the masking layer 13004. For example, the activator layer 13006 can include a DURAFIBER loaded with a hydrogel as described herein, such as a 50% sodium AMPS based hydrogel with a diacrylamide crosslinker with sodium iso-ascorbate as a reducing agent. A nitrite providing layer 13010 as described herein can be positioned below the activator layer 13006. The nitrite providing layer 13010 can be an integral part of the wound dressing 13000, or it can be provided as a separate component (e.g., provided in a packaging separate of the wound dressing 13000) to be utilized with the wound dressing 13000.

### Nitric Oxide Delivering Hydrogel-based Wound Dressing Formulations

As discussed earlier, under normal atmospheric conditions, nitric oxide (NO) is a short-lived, unstable gaseous substance. The instability is due to the unpaired electron of nitrogen and as an unstable substance with an unpaired electron, nitric oxide can be described as a free radical. However, compared with typical free radicals (for example, hydroxyl radical or superoxide), whose life-time is in the order of milliseconds, nitric oxide is relatively stable and is typically converted to a more stable chemical species within seconds of its production. Thus, for example, if gaseous nitric oxide contacts air, it reacts rapidly with oxygen to generate nitrogen dioxide as follows:

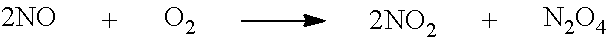

Moreover, although nitrogen dioxide (NO₂) may exert antimicrobial properties, it does not have vasodilating properties nor is it capable of activation of cell proliferation. It is therefore generally desirable to reduce the generation of nitrogen dioxide as far as possible in the acidification of nitrites such as by reducing the oxidation of dissolved nitric oxide (NO) by removing the oxygen from the body of the hydrogel where the acidification of nitrite takes place.

Under specific conditions, for instance when in a pure gaseous state, NO can be stored without significant losses for a very long time. NO is a very hydrophobic compound and its solubility in water is therefore limited. The maximum solubility of NO in water achievable under normal conditions is approximately 1.7 mM, the solubility being similar to that of oxygen. The oxidation of dissolved nitric oxide by dissolved oxygen occurs in aqueous solutions. Nevertheless, given the rate constants and low concentrations of dissolved NO and O₂ this reaction is considerably less rapid than in the gaseous state, where the concentration of oxygen is very high. In particular, some embodiments disclosed herein advantageously reduce the oxidation of dissolved nitric oxide (NO) by removing the oxygen from the body of the hydrogel where the acidification of nitrite takes place.

One of skill in the art will understand that the nitric oxide generating or nitrite providing layers described herein, particularly in relation to FIGS. 1-5, 11A-11E, 12, and 13 described above and elsewhere in the specification, may include both a nitric oxide source element (such as a nitrite providing element as disclosed herein) and an activator (such as an acid providing element as disclosed herein), for example, a nitrite providing layer, such as a nitrite providing layer as disclosed herein and an activator layer, such as an acid providing layer as disclosed herein. Interaction between a nitrite providing element and an acid providing element may result in the formation of nitric oxide suitable for delivery to a wound via suitable means. One of skill in the art will further understand that the following formulations may be utilized with any of the embodiments described herein, such as the wound dressings and apparatuses of FIGS. 1-5, 11A-11E, 12, and 13.

As will be understood by one of skill in the art, hydrogels may be constructed from various polymers, for example polyethylene glycols (PEG), hydrophilic polyurethanes, polyvinyl alcohols, polyvinypyrrolidone, or other suitable polymers. Such hydrogels may be cross-linked via suitable multifunctional reagents, condensation, polymerization, irradiation, physical cross-linking, or via other suitable means. One of skill in the art will understand that any suitable cross-linking molecule may be used, such as N,N′-methylenebisacrylamide in the case of polyethylene glycol hydrogels. As will further be understood by one of skill in the art, conventional crosslinking agents are used to provide the necessary mechanical stability and to control the adhesive properties of the composition. In certain embodiments, crosslinkers may include tripropylene glycol diacrylate, ethylene glycol dimethacrylate, alkoxylated triacrylate, polyethylene glycol diacrylate (PEG400 or PEG600), and/or methylene bis acrylamide. One of skill in the art will also understand that acidic functionality may be introduced to such hydrogel systems to generate nitric oxide. For example, suitable reagents to introduce such functionality might include silane coupling agents (such as those provided by Gelest). In some embodiments, these silane coupling agents may provide triethoxy silane or multi-silanol end groups which will react with surface hydroxyl groups, which are abundant on cellulose based substrates, resulting in pendant side groups bearing carboxylate/carboxylic acid or sulfonate/sulfonic acid groups. Carboxylate/carboxylic acid and sulfonate/sulfonic acid groups will be described in more detail below.

In some embodiments, an acid providing layer such as a hydrogel-based wound dressing formulation may comprise a copolymer wherein the monomers are functionalized with covalently linked acidic functional groups having the formula I:

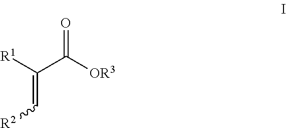

wherein R¹ is selected from the group consisting of optionally substituted C₁₋₄ alkyl, —CH₂COOR₃, —CH₂SO₂R³, and —CH₂P(O)(OR³)₂; R² is selected from the group consisting of optionally substituted C₁₋₄ alkyl, —COOR₃, and —SO₂R³; —PO(OR³)₂; and R³ is selected from the group consisting of —H and optionally substituted C₁₋₄ alkyl and a cation.

In some embodiments of formula I, R³ may be a cation such as a sodium ion, potassium ion, lithium ion, ammonium ion, trimethyl ammonium ion, or any other suitable cation.

In some embodiments of formula I, the monomer may be a crotonic acid, itaconic acid, fumaric acid, maleic acid, vinyl phosphonic acid, vinyl sulfonic acid and salts thereof, or any other suitable monomer.

As disclosed in the above embodiment and below, a suitable monomer that presents both an unsaturation (i.e. a portion capable of being incorporated into an acrylic copolymer via UV or radically initiated polymerization) and sufficient acidity may cause the efficient generation of nitric oxide from nitrites. Furthermore, these alternative monomers may offer advantages in terms of some examples offering multifunctionality. In particular, the carboxylic acid monomers described herein may offer more than one carboxylic acid group per unit of the monomer and therefore more acidic functional groups may be presented in an equivalent amount of material as compared to an acrylic acid (AA) or 2-acrylamido 2-methylpropanesulfonic (AMPS) based system. In some embodiments, such an arrangement may offer advantages in the yield of the desired NO product or other elements of controlling the profile of the product generated upon activation with a source of nitrite.

One of skill in the art will understand that any suitable initiator in suitable quantities may be used to initiate polymerization of the hydrogels disclosed herein, for example: 2,2-Dimethoxy-2-phenylacetophenone, ferrous sulfate heptahydrate, hydrogen peroxide, potassium bisulfite, potassium persulfate, thermal thiosulfate or mixtures thereof. Further details regarding initiators may be found in U.S. Patent No. U.S. Pat. No. 4,581,821, which is hereby incorporated by reference. Further examples of photo-initiators include 1-hydroxycyclohexyl phenyl ketone and 2-hydroxy-2-methylpropiophenone. Sources of energy for initiation of polymerization may be any suitable sources such as described herein, for example: light (such as ultraviolet), radiation (such as gamma), heat, chemical, or any suitable source of energy.

In certain embodiments, the hydrogel-based wound dressing formulation may comprise a copolymer wherein the monomers are functionalized with covalently linked reductant functional groups having the formula II:

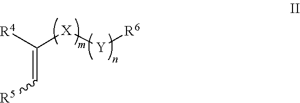

wherein R⁴ and R⁵ are independently selected from the group including-H, optionally substituted C₁₋₄ alkyl, optionally substituted C₆₋₁₀ aryl, and optionally substituted C₆₋₁₀ aralkyl; wherein X is selected from the group consisting of optionally substituted C₁₋₄ alkyl, —CH₂COO—, —COO—, —CH₂SO₂—, —SO—, —SO₂—, —CH₂CONH—, —CONH—, —P(O)(O)—, and —CH₂P(O)(O)—; wherein Y is selected from the group consisting of optionally substituted C₁₋₄ alkyl, optionally substituted C₃₋₇ carbocyclyl, PEG-chain, sugar unit, optionally substituted C₆₋₁₀ aryl, and optionally substituted C₆₋₁₀ aralkyl; R⁶ is a reductant functional group selected from the group consisting of iodide anion, butylated hydroquinone, tocopherol, butylated hydroxyl-anisole, butylated hydroxytoluene 2,3-dihydoxyphenyl group, 3,4-dihydroxyphenyl group, beta-carotene or any suitable group. In certain embodiments, m may be an integer from 1 to 2; and n may be an integer from 0 to 4. In some embodiments, m may range from 2 to 10 or more, while n may range from 4 to 10 or more. In certain examples, m or n may be any suitable integer.

In some embodiments of formula II, the covalently linked reductant functional group of the monomer may include 3,4-dihydroxyphenyl or 2,3-dihydroxyphenyl group or any suitable functional group.

As disclosed in the above embodiments and elsewhere herein, a suitable monomer that presents a covalently linked functionality that can act as a reducing agent in the NO generation chemistry, whilst remaining attached to the hydrogel structure may present advantages from a safety/regulatory viewpoint.

In certain embodiments, nitric oxide can be produced by chemical reduction of nitrous acid. Many different reducing agents may be used to reduce nitrous acid, physiologically acceptable examples of such reducing agents include but are not limited to: iodide anion, ascorbic acid, butylated quinone, tocopherol, or any suitable reducing agents. Nitrous acid is a weak acid with pKa 3.4 and therefore at pH ~3.4, nitrous acid exists as an equimolar mixture of nitrous acid (HNO₂) and nitrite (NO₂⁻¹). At higher pH values the equilibrium shifts in favor of the nitrite anion; at lower pH the equilibrium shifts in favor of nitrous acid. Since the nitrous acid can be chemically reduced to nitric oxide the efficiency of converting nitrite into nitric oxide may increase with decreasing pH. Therefore, in some embodiments, whilst at pH ~6 the rate of such conversion is negligible, it proceeds slowly at pH ~5 and is very rapid at pH <4 and especially faster at pH <3.

In embodiments, hydrogel systems based on predominantly the sodium salt of AMPS containing relatively small amounts of either the strongly acidic AMPS acid and/or the weakly acidic AA may provide a sufficiently acidic environment. Furthermore, non-thiol reducing agents that are not acids with pKas between about 1 to 4 may be employed as the reductant in these systems. The reductant may be present in any suitable component of the wound dressing systems. Examples of suitable reducing agents include but are not limited to iodide anion, butylated hydroquinone, hydroquinone, hydroquinone 53 ariants, tocopherol, butylated hydroxyanisole, butylated hydroxytoluene, beta-carotene, ascorbate, ascorbate variants, iso-ascorbate, iso-ascorbate variants, and any other suitable reducing agent. The reductant is typically present in concentrations of about: 0.01% to 5% (w/w), 0.01% to 0.1% (w/w), 0.05% to 0.1% (w/w), 0.1% to 0.2% (w/w), 0.3% to 0.4% (w/w), 0.1% to 5% (w/w), 0.5% to 4% (w/w), 1% to 3% (w/w), or around 2% (w/w) based on the dressing. The inclusion of a reducing agent covalently linked in the monomer as disclosed in the above embodiment may advantageously offer safety and regulatory benefits.

In some embodiments, a hydrogel-based wound dressing formulation such as described herein comprises monomers according to formula I and/or formula II. In certain embodiments, a hydrogel-based wound dressing formulation as described herein further comprises oxygen scavengers, such oxygen scavengers may include glucose, glucose peroxidase, iron-based scavengers such as nano-iron particles, boron-based scavengers such as nano-boron particles, and electrolyes such as sodium chloride. The oxygen scavengers may be incorporated into the formulation via any suitable means, for example via dissolving, absorption, adsorption, and/or attachment to the polymer structure. Such oxygen scavengers may require protection from an aqueous environment, such as any of the hydrogels disclosed herein and therefore may be sealed from the aqueous portion or incorporated into a polymeric composite within the body of the gel. Such oxygen scavengers may also advantageously remove oxygen from the hydrogel during storage over a period of time, thereby potentially improving the efficiency of nitric oxide production. In certain embodiments, the dressing may be manufactured in an inert environment to prevent oxygen ingress during manufacture. Such a dressing may also be sealed such that no oxygen ingress occurs prior to application of the dressing.

### Hydrogel-based Wound Dressing System:

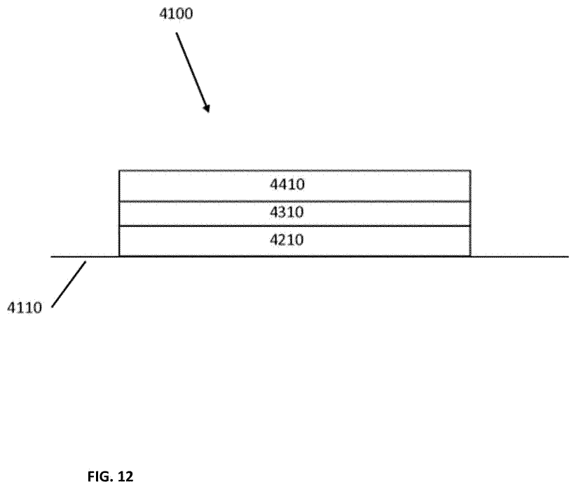

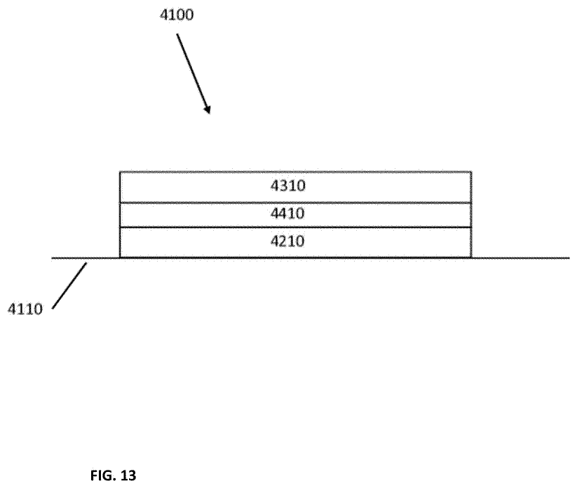

As depicted in FIGS. 12 and 13, in some embodiments a hydrogel-based wound dressing system 4100 may be placed over a wound and/or intact skin such as in the periwound area, and may include a first acid providing layer 4210 containing a copolymer of monomers with covalently linked multifunctional groups wherein the monomers are functionalized with covalently linked acidic functional groups having the formula I, wherein R¹ may be —CH₂SO₂R³ or —CH₂P(O)(OR³)₂ or any suitable group; R² may be —SO₂R³ or —PO(OR³)₂ or any suitable group; and R³ may be —H and optionally substituted C₁₋₄ alkyl or any suitable group.

In certain embodiments, a second acid providing layer 4310 may be positioned above the first acid providing layer and contain a copolymer of monomers with covalently linked multifunctional groups wherein the monomers are functionalized with covalently linked acidic functional groups having the formula I, wherein R¹ may be optionally substituted C₁₋₄ alkyl, —CH₂COOR₃; R² may be optionally substituted C₁₋₄ alkyl, and —COOR₃; and R³ may be —H and optionally substituted C₁₋₄ alkyl and a cation. One of skill in the art will understand that the acid providing layers may be in any order, such as first on top of second or second on top of first.

In some embodiments, the hydrogel-based wound formulation and dressing system may be free of water or substantially free of water. For example, the acid providing layers described in the embodiments herein may be in the format of either a xerogel (hydrogel is allowed to change its dimensions during the slow removal/reduction of water content) or an aerogel (the structure of the hydrogel is little impacted as the water is rapidly removed via supercritical or freeze drying etc.). In some embodiments, the hydrogel-based wound formulation and dressing system may have a moisture content (e.g., water content) of less than about 1%, less than about 2%, less than about 3%, less than about 4%, or less than about 5%.

In certain embodiments, the acidic component in the wound dressing system in the form of a xerogel/aerogel may afford a reduction in the weight of the dressing system, which may be advantageous to a wound dressing product (lighter, lower profile product). The physical properties of the system may also be advantageous (for example, different rates of absorbency may also reflect a unique NO production profile). As explained above, in certain embodiments, the hydrogel-based wound dressing system 4100, may include a first acid providing layer 4210 containing a copolymer of monomers with covalently linked multifunctional groups; and a second acid providing layer 4310 above first acid providing layer, containing a copolymer of monomers with covalently linked multifunctional groups. In particular, embodiments of the present disclosure allow for a blend or combination of the two acidic monomers. For example, such an embodiment may be achieved through formulation of a single gel layer or via the construction of gel layers that consist of “stacks” or patterning whereby the different layers/regions consist of differing acidic nature types. Such a stacked arrangement may include 2, 3, 4, 5 or more different acid providing layers. The stack may include 2, 3, 4, 5, 6, or more acid providing layers. In certain embodiments, one or more layers may be non-acidic, for example 1, 2, 3, 4, 5, or 6 or more non-acidic layers.

In embodiments, the release profile may be controlled by creating a hydrogel-based wound dressing system that consists of appropriate amounts of both strong and weak acids attached to the polymeric structure. In certain embodiments, the hydrogel may be constructed from layers or patterned regions comprising the strong and/or weak acids. Therefore, different strength acids may be layered or patterned throughout the profile of the finished hydrogel sheet. For example, a strong acid environment may be positioned at the wound facing side to provide a very rapid NO release (efficient nitrite conversion) and a weaker acid as a subsequent further layer removed from the wound which would afford a slower conversion of nitrite to NO (and therefore provide a more sustained element of the release profile as the nitrite solution provided is drawn up through the hydrogel stack).

Returning to FIGS. 12-13, the hydrogel-based wound dressing system 4100 may include a nitrite providing layer 4410. As depicted in the figures, the nitrite providing layer may be above or below the acid providing layer or layers. In some embodiments, the source of nitrite may be a suitable material layer that includes alkali metal nitrites and/or alkaline earth metal nitrites. In certain embodiments, the nitrites may include: LiNO₂, NaNO₂, KNO₂, RbNO₂, CsNO₂, FrNO₂, Be(NO₂)₂, Mg(NO₂)₂, Ca(NO₂)₂, Sr(NO₂)₂, Ba(NO₂)₂, Ra(NO₂)₂ or any other suitable nitrite. In certain embodiments, the nitrite providing layer may contain sodium nitrite. In some embodiments, other sources of nitrite ions may be nitrate ions derived from alkali metal or alkaline earth metal salts capable of enzymic conversion to nitrite. For example, LiNO₃, NaNO₃, KNO₃, RbNO₃, CsNO₃, FrNO₃, Be(NO₃)₂, Mg(NO₃)₂, Ca(NO₃)₂, Sr(NO₃)₂, Ba(NO₃)₂, Ra(NO₃)₂, or any other suitable molecule.

In certain embodiments, the acid providing layers such as described above and elsewhere herein may contain covalently bound acidic functional groups, and are therefore capable of generating nitric oxide when contacted with a suitable source of nitrite such as an alkali metal nitrite. In embodiments, covalent functionalization of suitable polymeric wound dressing materials, surface coating, or plasma functionalization may render said dressing materials suitably acidic in nature to efficiently convert nitrite to NO.

Hydrogels with Various Crosslinkers with and without an Antioxidant and/or Reducing Agent

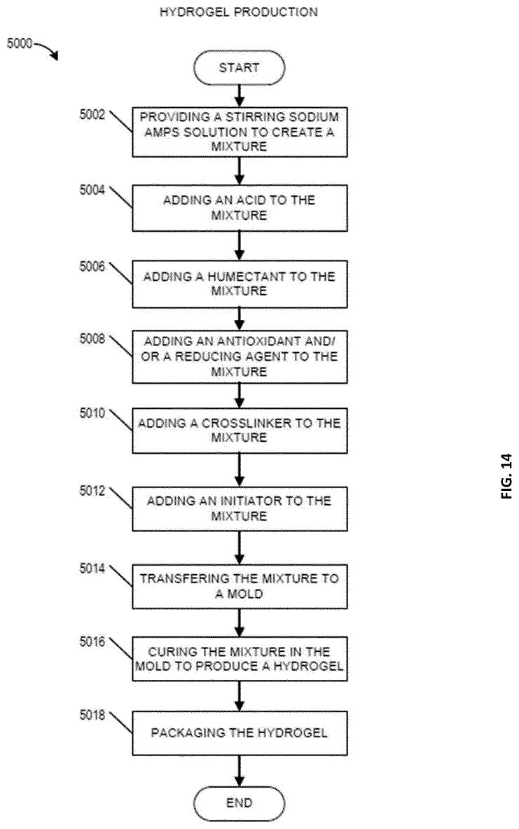

FIG. 14 shows a process 5000 for hydrogel production according to some embodiments. An activator layer of a wound dressing as described herein may comprise a hydrogel produced by the process 5000. While multiple process steps are shown in FIG. 14, it is to be understood that not all process steps are required and some may be optional. Furthermore, it is to be understood that the order in which the process steps of FIG. 14 take place may be modified or rearranged, and additional process steps not shown may be added at any suitable step in the process. As shown, the process 5000 for hydrogel production may comprise a step 5002 of providing a stirring sodium AMPS solution to create a mixture, a step 5004 of adding an acid to the mixture, a step 5006 of adding a humectant to the mixture, a step 5008 of adding an antioxidant and/or a reducing agent to the mixture, a step 5010 of adding a crosslinker to the mixture, a step 5012 of adding an initiator to the mixture, a step 5014 of transferring the mixture to a mold, a step 5016 of curing the mixture in the mold to produce a hydrogel, and a step 5018 of packaging the hydrogel.

As shown in FIG. 14, the process 5000 for producing a hydrogel may comprise the step 5002 of providing a stirring sodium AMPS solution (e.g., an aqueous Sodium 2-Acrylamido-2-methyl-1-propanesulfonate solution) to create a mixture. In some embodiments, the sodium AMPS solution comprises a 50% w/w solution, however other concentrations may be used. In some embodiments, a vessel comprising the stirring sodium AMPS solution may be surrounded by a water bath maintained between about 10 C to about 15 C, at about 10 C, or at about 15 C. In some embodiments, the stirring sodium AMPS solution may be stirred to form a significant vortex without sucking in air.

As further shown in FIG. 14, the process 5000 for producing a hydrogel may additionally comprise the step 5004 of adding an acid to the mixture. The acid may comprise acrylic acid, 2-Acrylamido-2-methyl-1-propanesulfonic acid (AMPS acid), any appropriate acid that can be reacted into the hydrogel structure, and/or a combination thereof. In some embodiments, the acid may comprise any of the acids described herein and/or a combination of any of the acids described herein.

As further shown in FIG. 14, the process 5000 for producing a hydrogel may additionally comprise the step 5006 of adding a humectant to the mixture. The humectant may comprise glycerol, propylene glycol, any of the humectants described herein, and/or any combination thereof. In some embodiments, the step 5006 of adding a humectant to the mixture may be omitted.

As further shown in FIG. 14, the process 5000 for producing a hydrogel may additionally comprise the step 5008 of adding an antioxidant and/or a reducing agent to the mixture. The antioxidant and/or reducing agent may comprise sodium iso-ascorbate, iodide anion, butylated hydroquinone, hydroquinone, hydroquinone 57 ariants, tocopherol, butylated hydroxyanisole, butylated hydroxytoluene, beta-carotene, ascorbic acid, potassium iodide, ascorbate, ascorbate variants, iso-ascorbate, iso-ascorbate variants, any other suitable reducing agent, and/or any of the antioxidants and/or reducing agents described herein. The antioxidant and/or a reducing agent may be present in concentrations of about: 0.01% to 5% (w/w), 0.01% to 0.1% (w/w), 0.05% to 0.1% (w/w), 0.1% to 0.2% (w/w), 0.3% to 0.4% (w/w), 0.1% to 5% (w/w), 0.5% to 4% (w/w), 1% to 3% (w/w), or around 2% (w/w). In some embodiments, a ratio of the amount of antioxidant and/or reducing agent to the amount of acid may be about 1:2, about 1:1.5, about 1:1, about 1:0.9, about 1:0.8, about 1:0.7, about 1:0.6, about 1:0.5, about 1:0.4, about 1:0.3 or between about 1:2 and about 1:0.3. In some embodiments, the step 5008 of adding an antioxidant and/or a reducing agent may be omitted. In some embodiments, the step 5008 of adding an antioxidant and/or a reducing agent to the mixture may be performed near the end of the process 5000, such as after the step 5010 of adding a crosslinker to the mixture or after the step 5012 of adding an initiator to the mixture and before the step 5014 of transferring the mixture to a mold. In such embodiments, adding the antioxidant and/or reducing agent can advantageously prevent or reduce pre-gelling of the mixture.

As further shown in FIG. 14, the process 5000 for producing a hydrogel may additionally comprise the step 5010 of adding a crosslinker to the mixture. In some embodiments, the crosslinker may comprise an acrylate, dimethacrylate, and/or an acrylamide crosslinker. In some embodiments, the acrylate crosslinker may comprise PEG diacrylate Mn 575. In some embodiments, the dimethacrylate crosslinker may comprise PEG dimethacrylate Mn 550. In some embodiments, the acrylamide crosslinker may comprise Piperazine diacrylamide. In some embodiments, the crosslinker may comprise tripropylene glycol diacrylate, ethylene glycol dimethacrylate, alkoxylated triacrylate, polyethylene glycol diacrylate (PEG400 or PEG600), and/or methylene bis acrylamide. In some embodiments, the crosslinker may comprise any of the crosslinkers described herein and/or any combination of the crosslinkers described herein. In some embodiments, the crosslinker may reduce and/or prevent hydrolysis of the hydrogel. For example, a dimethacrylate and/or acrylamide crosslinker may reduce and/or prevent the hydrolysis of a hydrogel comprising said crosslinker(s). In some embodiments, the crosslinker that reduces and/or prevents hydrolysis of the hydrogel may improve the mechanical stability of the hydrogel.

As further shown in FIG. 14, the process 5000 for producing a hydrogel may additionally comprise the step 5012 of adding an initiator to the mixture. The initiator may comprise a photoinitiator, a thermal initiator, any of the initiators described herein, or any initiator configured to induce polymerization. In some embodiments, the initiator may comprise 2-hydroxy-2-methylpropiophenone, 2,2-Dimethoxy-2-phenylacetophenone, ferrous sulfate heptahydrate, hydrogen peroxide, potassium bisulfite, potassium persulfate, thermal thiosulfate, and/or mixtures thereof. Further details regarding initiators may be found in U.S. Patent No. U.S. Pat. No. 4,581,821, which is hereby incorporated by reference. Further examples of photoinitiators include 1-hydroxycyclohexyl phenyl ketone and 2-hydroxy-2-methylpropiophenone.

As further shown in FIG. 14, the process 5000 for producing a hydrogel may additionally comprise the step 5014 of transferring the mixture to a mold. The process 5000 may additionally comprise the step 5016 of curing the mixture in the mold to produce a hydrogel. The mold may comprise a Teflon coated aluminum mold or any suitable mold for releasably disengaging the hydrogel after being formed. Sources of energy for initiation of polymerization/curing may be any suitable sources such as described herein, for example: light (such as ultraviolet), radiation (such as gamma), heat, chemical, or any suitable source of energy.

As further shown in FIG. 14, the process 5000 for producing a hydrogel may additionally comprise the step 5018 of packaging the hydrogel. In some embodiments, packaging the hydrogel may comprise packaging a wound dressing comprising the hydrogel. In some embodiments, packaging the hydrogel may be performed in the absence of oxygen and/or in a low oxygen environment. In some embodiments, packaging the hydrogel may be performed under reduced pressure and/or vacuum. In some embodiments, packaging the hydrogel may be performed in an inert environment and/or in the presence of an inert gas (e.g. Argon and/or Nitrogen). In some embodiments, packaging used for packaging the hydrogel may be air-tight and/or may prevent oxygen outside the packaging from interacting with the hydrogel and/or wound dressing until the packaging is opened. In some embodiments, the packaging used for packaging the hydrogel may exclude oxygen. In some embodiments, oxygen-scrubbing may be employed to remove oxygen from the packaging. In some embodiments, the packaging may comprise foil packaging. In some embodiments, the packaging may comprise an oxygen scavenger packaged with the hydrogel and/or wound dressing comprising the hydrogel. The oxygen scavenger may remove oxygen from the wound dressing and/or its components, such as the hydrogel/activator layer and nitrite providing layer, once sealed in the packaging. In some embodiments, the oxygen scavenger may remove oxygen from within the packaging once sealed. In some embodiments, the sodium iso-ascorbate in a hydrogel comprising sodium iso-ascorbate may scavenge oxygen and/or reduce the oxygen content of the wound dressing and/or its components, such as the nitrite providing layer and/or the activator layer. In some embodiments, the sodium iso-ascorbate in a hydrogel comprising sodium iso-ascorbate may scavenge a residual oxygen content within the packaging. In some embodiments, a hydrogel comprising sodium iso-ascorbate may comprise a sacrificial amount of sodium iso-ascorbate to scavenge and/or reduce oxygen in and/or around the wound dressing and its components as described herein.

In some embodiments, the process 5000 for producing a hydrogel may additionally comprise a step of adding an oxygen scavenger to the mixture before transferring the mixture to a mold as in step 5014. The oxygen scavenger may comprise glucose, glucose peroxidase, an iron-based scavenger, any of the oxygen scavengers described herein, and/or any mixture thereof. In some embodiments, the oxygen scavenger may comprise an antioxidant as described herein, such as sodium iso-ascorbate.

In some embodiments, one or more steps of process 5000 may be performed in an environment that minimizes the oxygen content of the hydrogel produced by the process 5000. In some embodiments, one or more steps of process 5000 may be performed in the absence of oxygen and/or in a low oxygen environment. In some embodiments, one or more steps of process 5000 may be performed under reduced pressure and/or vacuum. In some embodiments, one or more steps of process 5000 may be performed in an inert environment and/or in the presence of an inert gas, such as Argon and/or Nitrogen. In some embodiments, a hydrogel after being produced by the process 5000 may be degassed and/or undergo reduced pressure cycling/vacuuming. In some embodiments, a nitrite providing layer and/or components thereof (e.g., a sodium nitrite solution that saturates a mesh of a nitrite providing layer) as described herein may be degassed and/or undergo reduced pressure cycling/vacuuming. In some embodiments, a wound dressing as described herein may be degassed and/or undergo reduced pressure cycling/vacuuming. In some embodiments, one or more steps of process 5000 may be performed in a low light environment and/or an environment that minimizes curing of the mixture until desired at step 5016 of the process 5000. In some embodiments, a hydrogel produced by process 5000 may comprise a dissolved oxygen content less than about 200 ppb, about 250 ppb, about 300 ppb, about 400 ppb, about 500 ppb, or about 1000 ppb. In some embodiments, a wound dressing comprising a hydrogel produced and packaged by process 5000 with minimized oxygen exposure/content may increase a nitric oxide production capability of the wound dressing. In some embodiments, a wound dressing comprising a hydrogel produced and packaged by process 5000 with minimized oxygen exposure/content may increase a ratio of nitric oxide to nitrogen dioxide produced by the wound dressing (e.g., when the wound dressing is placed over a wound).

In some embodiments, a hydrogel produced by process 5000 may comprise a Tan D value as evaluated by Rheometry under about 0.35, about 0.45, about 0.55, about 0.65, and/or under about any Tan D value that provides for optimum hydrogel properties. In some embodiments, a wound dressing comprising a hydrogel produced by process 5000 may comprise a Tan D value as evaluated by Rheometry under about 0.35, about 0.45, about 0.55, about 0.65, and/or under about any Tan D value that provides for optimum wound dressing properties. In some embodiments, a wound dressing comprising a hydrogel produced by process 5000 may comprise a Tan D value as evaluated by Rheometry that remains under about 0.35, about 0.45, about 0.55, about 0.65, and/or under about any Tan D value that provides for optimum wound dressing properties when packaged in a packaging and until the packaging is opened. In some embodiments, a wound dressing comprising a hydrogel produced by process 5000 may remain substantially colorless when packaged in a packaging until the packaging is opened.

A hydrogel produced by the process 5000 may be used in any of the wound dressings described herein. In some embodiments, an activator layer (and/or acid providing layer) as described herein may comprise a hydrogel as produced by the process 5000. In some embodiments, a wound dressing comprising a hydrogel as produced by the process 5000 may additionally comprise a nitrite providing layer as described herein. In some embodiments, the hydrogel produced by the process 5000 may be configured to donate a proton to the nitrite providing layer to produce nitric oxide as described herein. In some embodiments, the nitrite providing layer may comprise sodium nitrite. In some embodiments, the nitrite providing layer may comprise a mesh. In some embodiments, the nitrite providing layer may comprise a saturated polypropylene mesh. In some embodiments, a wound dressing comprising a hydrogel as produced by the process 5000 may additionally comprise any of the other layers and/or aspects of the wound dressings described herein, such as an acquisition distribution layer, a cover layer, and/or a masking layer.

As will be understood by one of skill in the art, any amount of the aspects/ingredients as described in the process 5000 for producing a hydrogel may be utilized to produce a hydrogel with desired properties. For example, in some embodiments a standard amount or double, triple, or quadruple the required amount of crosslinker may be used in the process 5000. As another example, in some embodiments a standard amount or double the required amount of initiator may be used in the process 5000. As another example, in some embodiments a standard amount or double, triple, or any excess amount of antioxidant and/or reducing agent may be used in the process 5000. Furthermore, a hydrogel produced by the process 5000 may be of any thickness, length, and width as desired. In some embodiments, a hydrogel may comprise one, two, three, or more layers, which may be prepared by consecutively transferring a mixture to a mold and curing one layer on top of another, or by placing a hydrogel produced on top of another. In some embodiments, each layer of a hydrogel comprising multiple layers may comprise the same or different formulations.

In some embodiments and as described in the process 5000 for producing a hydrogel, the hydrogel may comprise an antioxidant and/or reducing agent. The antioxidant and/or reducing agent may promote the generation of nitric oxide by a wound dressing comprising the hydrogel. In some embodiments, a wound dressing comprising a hydrogel with an antioxidant and/or reducing agent may produce an amount of nitric oxide that is greater than the amount of nitric oxide produced by a wound dressing comprising the hydrogel without the antioxidant and/or reductant. In some embodiments, a wound dressing comprising a hydrogel with an antioxidant and/or reducing agent may produce 2 times, 5 times, 10 times, or 20 times as much nitric oxide than a hydrogel without the antioxidant and/or reducing agent. In some embodiments, a wound dressing comprising a hydrogel with an antioxidant and/or reducing agent may produce a ratio of nitric oxide to nitrogen dioxide that is greater than a ratio of nitric oxide to nitrogen dioxide produced by a wound dressing comprising the hydrogel without the antioxidant and/or reductant. In some embodiments, a wound dressing comprising a hydrogel with an antioxidant and/or reducing agent may produce a ratio of nitric oxide to nitrogen dioxide that is at least 1:1, 2:1, 3:1, 4:1 or 5:1. As described herein, a wound dressing producing more nitric oxide than nitrogen dioxide may be desirable.

One of skill in the art will understand that the hydrogels produced and/or packed as described above may be utilized with any of the embodiments described herein, such as the wound dressings and apparatuses of FIGS. 1-5, 11A-11E, 12, and 13.

Examples of Hydrogels with Various Crosslinkers without an Antioxidant and/or Reducing Agent

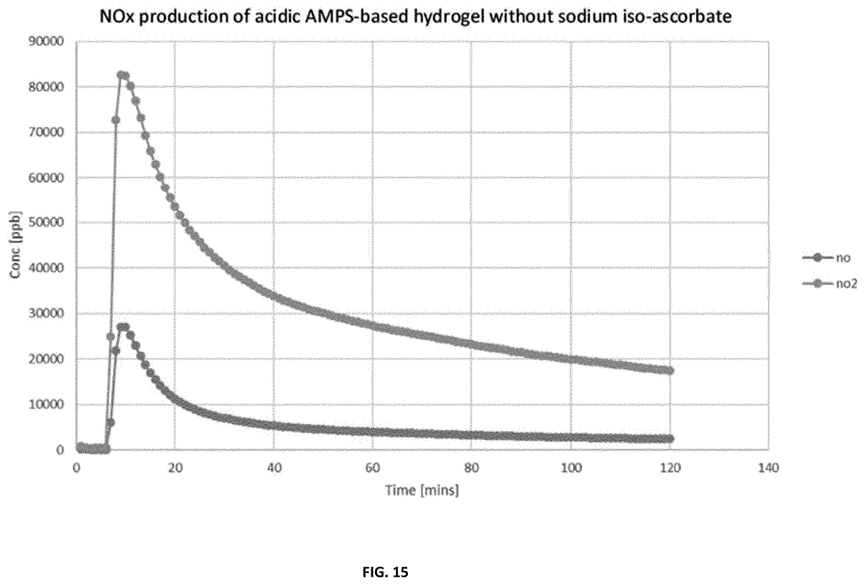

FIG. 15 shows data on nitric oxide and nitrogen dioxide produced by a hydrogel without the reducing agent sodium iso-ascorbate, through time. Described below is an example process for preparing the hydrogels used to produce the data of FIG. 15.

### Method

Into a suitably sized beaker, equipped with an overhead propeller type stirrer and surrounded by a water bath controlled at 15 C, was added 50% aqueous Sodium 2-Acrylamido-2-methyl-1-propanesulfonate solution (Na AMPS). The following components were added with stirring to form a significant vortex without sucking in air: pre-ground 2-Acrylamido-2-methyl-1-propanesulfonic acid (AMPS acid) and Glycerol (slow addition), after the AMPS acid had dissolved. This was followed by addition of the selected cross linker (listed in the materials section) then 2-hydroxy-2-methylpropiophenone photoinitiator. To facilitate the dissolution of the less soluble Piperazine diacrylamide in a reasonable time period, this crosslinker was added prior to the AMPS acid and Glycerol which were only added after its dissolution. The mixture was left to stir for 10-20 minutes. All hydrogel mixes used the standard 0.5 SA formulation.

The prepared prepolymer mixture was formed into 3 mm thick hydrogel sheets by transferring 41.2 ml (53.4 g) via a 50 ml Eppendorf pipette to a 11×11×3 cm Teflon coated aluminium mold. The mold was first placed onto a level shelf in a UVC cabinet prior to transferring the prepolymer mixture to the mold which was then exposed to a UVC flood light (UVC output: ~1.4 mW/cm2) for 60 seconds. The cured sheet was removed from the mold and placed between silicone release paper within a sealed bag and assigned a batch reference.

The hydrogel sheets were cut into either 2.5 cm disks for Rheometry or into 3×3 cm squares for Nox determination (for NO and NO₂ data as shown in FIG. 15). The gel disks or squares were packed in triplicate, in foil packaging and sealed either under vacuum, atmospheric air, or nitrogen as indicated. The vacuum, nitrogen, and air sealed pouches were either subjected to gamma sterilisation or left unsterilized and stored at 25 C/60% RH or 40 C/75% RH. At various time points the gels were removed from storage and evaluated in triplicate for Rheometry and Nox output determination.

Chemiluminescence Nox Production Results: Hydrogel with/without Sodium Iso-Ascorbate

FIG. 15 shows chemiluminescence Nox production for a representative acidic AMPS-based hydrogel without sodium iso-ascorbate when contacted with a polypropylene mesh saturated in 1M aqueous sodium nitrite (NaNO_{2(aq)}).

As shown in FIG. 15, the hydrogel without sodium iso-ascorbate produced more nitrogen dioxide (NO₂) than nitric acid (NO) when contacted with the saturated mesh. More specifically, the hydrogel without sodium iso-ascorbate produced a peak of approximately 83000 ppb nitrogen dioxide and a peak of approximately 27000 ppb nitric acid, the peaks occurring at approximately the same time after contact with the saturated mesh (approximately after 3 minutes after contact as shown). Quantified, the ratio of nitric oxide to nitrogen dioxide produced at peak was approximately 1:3. As shown in FIG. 15, after peak production of nitrogen dioxide and nitric acid, the production of each reduced with time, with nitrogen dioxide production decreasing at a somewhat more rapid rate than nitric acid but with its production remaining higher than the production of nitric oxide.

### Chemiluminescence NO/NO2 Production Results: Sodium Nitrite and Sodium Isoascorbate Dispersed in PEG400

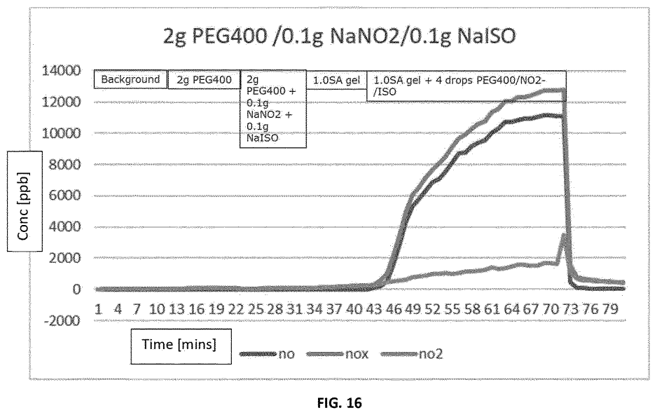

FIG. 16 shows an example of chemiluminescence NO/NO₂ production over a 79 minute time period for an example acidic hydrogel with 1.0 SA contacted with a suspension generated by dispersing 0.1 g NaNO₂ and 0.1 g NaISO (sodium iso-ascorbate) in 2 g of polyethylene glycol (PEG)400. As will be understood by one of skill in the art, the materials/constructions of FIGS. 16-18D are merely example for use with embodiments/examples described herein.

As shown in FIG. 16, during the 46-73 minute period corresponding to the application of the PEG400/NO₂/NaISO suspension to the hydrogel, more nitric oxide (NO) was produced than nitrogen dioxide (NO₂). These results indicated the suitability of PEG400 as an anhydrous water miscible carrier for a mixture of NaNO₂ and NaISO and demonstrated that contact between a PEG400/NO₂/NaISO suspension and the acidic hydrogel was capable of nitric oxide generation. Additionally, the time point of approximately 22-34 minutes corresponding to the measurement of nitric oxide generated from the PEG400/NO₂/NaISO suspension demonstrated that no detectable nitric oxide was generated from the PEG400/NO₂/NaISO suspension prior to contact with the acidic hydrogel.

### Antimicrobial Activity Results: Sodium Nitrite and Sodium Iso-Ascorbate Dispersed in PEG400

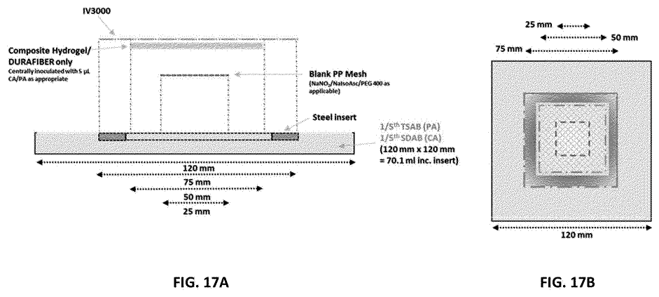

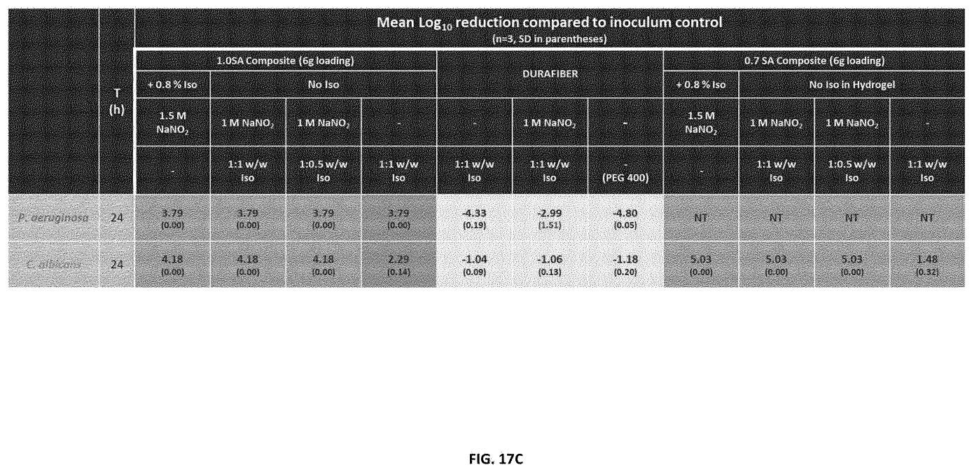

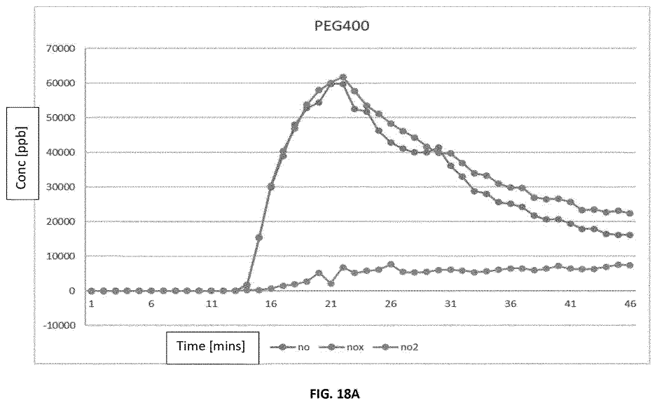

FIG. 17A-17C show examples of an evaluation of the antimicrobial activity of an example dressing composed of an IV3000 top layer, a hydrogel/DURAFIBER composite with 1.0 SA or 0.7 SA, and a polypropylene (pp) mesh embedded with a PEG400/NO₂/NaISO suspension. A 1.0 SA gel may be made of the following components (parts by weight of a 100 g batch): 63.32 g sodium AMPS; 0.0323 g piperazine diacrylamide; 5.436 g AMPS(H+); 31.19 g glycerol and 0.01629 g 2-hydroxy-2-methylpropiophenone. A 0.7 SA gel may be made of the following components (parts by weight of a 100 g batch): 65.28 g sodium AMPS; 0.0317 g piperazine diacrylamide; 3.923 g AMPS (H+); 30.74 g glycerol and 0.01756 g 2-hydroxy-2-methylpropiophenone.

FIG. 17A shows the placement of the layers of the dressing evaluated in a direct inoculation seal test model. An insert plate was prepared containing ⅕th trypticase soy agar (TSA) or ⅕th Sabouraud dextrose agar (SDA) with additional 3.3% bovine serum albumin (BSA). Overnight cultures of test organisms Pseudomonas aeruginosa (PA) and Candida albicans (CA) were adjusted to produce 4×10⁸ CFU/ml inoculum. 25 mm×25 mm 17 PP mesh (Blank) was placed centrally on the plate and dosed with 1 M NaNO₂ with either 1:1 W/W NaISO, or 1:0.5 NaISO, suspended in PEG400. 5 μl of appropriate inoculum was added (~2×10⁶ CFU total) directly onto the Composite/hybrid dressings, the composite dressings were 50 mm×50 mm in size and loaded with 6 g of 1.0 SA or 0.7 SA gel. A 75 mm×75 mm IV3000 top film was placed directly on top of the mesh to cover the steel insert and form a seal within 1-3 min of the dressing being applied. Treatment proceeded for 24 and 72 hour time points with 32° C. incubations. To assess microbial growth, the composite/IV3000/PP Mesh were added to 40 ml neutraliser (D/E broth), stomached for 2 min at high setting to recover, serially diluted counted on pour plates/Petrifilms. All dressing elements were prepared the same day as the experiment, and no gamma sterilisation was performed and no inert atmosphere was used

FIG. 17B shows the assembled dressing of FIG. 17A for the antimicrobial activity assessment when viewed from above and indicates the dimensions of the respective layers.

FIG. 17C summarizes the results of the microbial assessment in terms of the mean Log 10 reduction compared to inoculum control. The DURAFIBER control conditions without loading of hydrogel failed to inhibit proliferation of either test organism and were determined to be inactive. The conditions including a PP mesh embedded with 1 M NaNO₂ with either 1:1 W/W sodium iso-ascorbate, or 1:0.5 NaISO suspended in PEG400 showed equivalent results to the previously investigated formats where NaNO₂ and NaISO were not localized. The condition including 1.0 SA and hydrogel had greater innate activity than expected against Candida, but did not reduce to limit of detection, indicating the NaNO₂/NaISO mix attains target level activity in this model. Thus, relocating the NaISO from the hydrogel to the mesh where it would be colocalized with the NaNO₂ and maintain reduction of microbial growth was feasible.

Considering the results of the microbial activity assessment, an area of agar remained transparent when NaNO₂:NaISO ratio (w/w) was 1:1 on the mesh, however, this was less apparent when the ratio was reduced to 1:0.5.

### Chemiluminescence NO/NO2 Production Results: Sodium Nitrite and Sodium Iso-Ascorbate Dispersed in Various Anhydrous Water Miscible Carriers

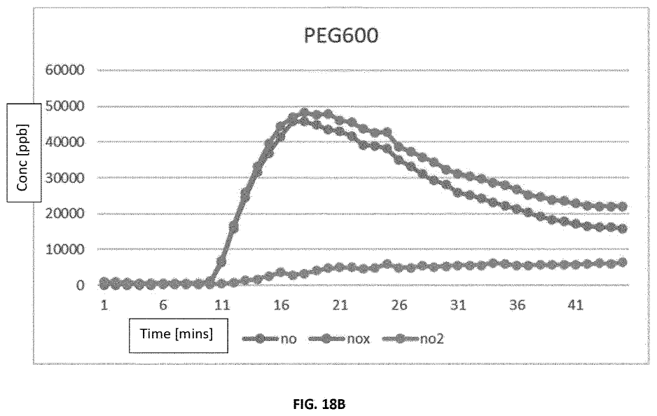

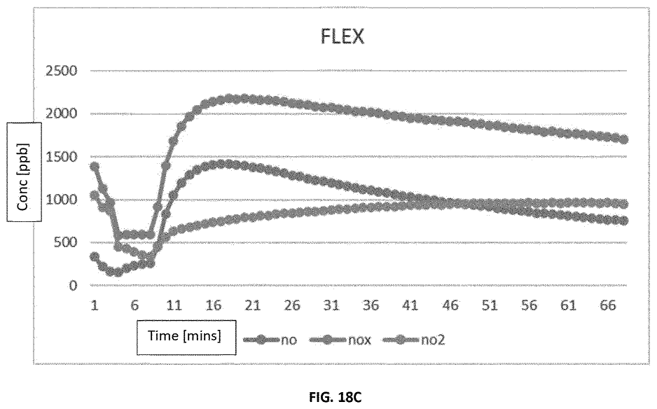

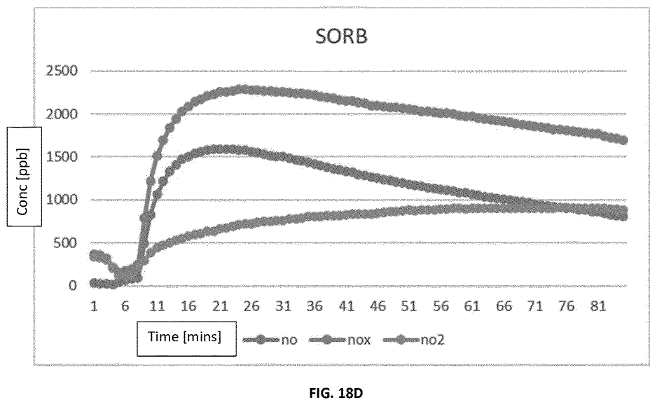

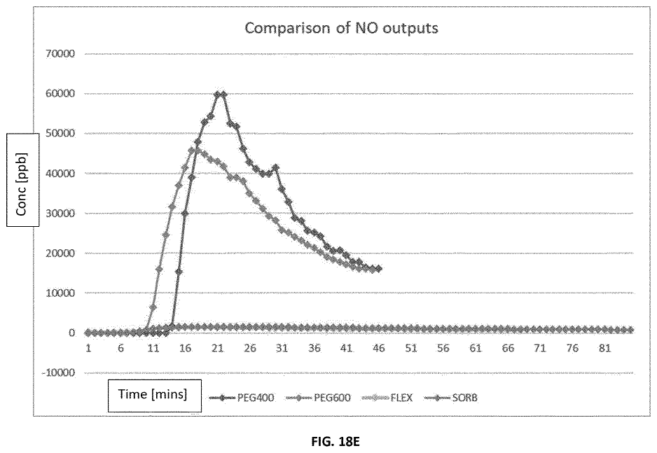

FIG. 18A-18D show example chemiluminescence NO/NO₂ production when 3×3 cm composites of DURAFIBER loaded with ~6 g 1.0 SA acidic hydrogel were contacted with 2.5×2.5 PP mesh embedded with 50 μL of a suspension generated by dispersing 0.1 g NaNO₂ and 0.1 g NaISO in various anhydrous water miscible carriers.

FIG. 18A shows nitric oxide production where PEG400 was used as the anhydrous water miscible carrier, FIG. 18B shows the results when PEG600 was used as the anhydrous water miscible carrier, FIG. 18C shows the results when a 50/50 w/w blend of PEG300 and PEG1500 (FLEX) was used as the anhydrous water miscible carrier, FIG. 18D shows the results when a 41/1 w/w blend of PEG400 and PEG4000 (SORB) was used as the anhydrous water miscible carrier, and FIG. 18E combines the results of FIG. 18A-18D on a single graph.

### Exemplary Wound Dressing Configurations and Formulations

Exemplary wound dressings can include a mesh component comprising a colocalized nitrite salt and antioxidant/reducing components. Exemplary wound dressings can include a mesh component comprising embedded, colocalized sodium nitrite and sodium iso-ascorbate. Exemplary wound dressings can include sodium iso-ascorbate monohydrate. Exemplary wound dressings can include anhydrous sodium iso-ascorbate. The mesh component can have a sodium nitrite dose of any number between about 0.5 M and about 2.5 M. Exemplary embodiments can include a 2.5×2.5 cm mesh loaded with any amount between 1-5 mg of each of a nitrite salt and an antioxidant/reducing agent suspended in a suitable carrier. Exemplary wound dressings can include a mesh component embedded with a suspension of 1:1 W/W sodium nitrite to sodium iso-ascorbate. Exemplary wound dressings can include a mesh component embedded with a suspension of 1:0.5 W/W sodium nitrite to sodium iso-ascorbate. Exemplary wound dressings can include a mesh component embedded with a suspension of 1:0.25 W/W sodium nitrite to sodium iso-ascorbate. Exemplary wound dressings can include a mesh component embedded with a suspension of sodium nitrite to sodium iso-ascorbate, wherein the sodium nitrite and sodium iso-ascorbate are present in any ratio ranging from 1:0.1-1:4 W/W sodium nitrite to sodium iso-ascorbate. Exemplary wound dressings can include a mesh component where the embedding suspension is a paste. Exemplary wound dressings can include a mesh component where the embedding suspension comprises a water soluble/miscible carrier. In some embodiments, the embedding suspension may be a paste. Exemplary wound dressings can include a mesh component where the embedding liquid/carrier is PEG200, PEG400, PEG600, or any blend of PEGs, triacetin, or glycerol. Exemplary wound dressings can include a mesh component where the embedding carrier is dried through the use of molecular sieves and other techniques known in the art. Exemplary wound dressings can provide greater stability to the reducing agent due to storage of the mesh component embedded with the reducing agent and nitrite providing components under dry conditions. Exemplary wound dressings can include reduction of undesirable colored species by storing mesh component embedded with the reducing agent and nitrite providing components under dry conditions. In exemplary wound dressings, the nitrite providing layer may be provided as a separate layer in a packaging separate of the packaging of the wound dressing. Exemplary wound dressings can provide more consistent nitric oxide generation than wound dressings where the reducing agent, such as sodium iso-ascorbate, iron sulfate, or a derivative of vitamin C, is stored under aqueous conditions and is subject to higher levels of degradation. Exemplary wound dressings with reducing agents kept under dry conditions before combination with the activating layer can have reduced degradation induced through free radical actions generated upon irradiation of aqueous solutions. Exemplary wound dressings can have favorable stability following sterilization compared to wound dressings where the reducing agent is stored under aqueous conditions. Exemplary wound dressings can include a cover layer, acquisition distribution layer and/or a masking layer. Exemplary wound dressings can include an IV3000 top film and border as the cover layer and a 17 gsm polypropylene mesh component. The polypropylene mesh can be any mesh within the range of 5 gsm to 200 gsm. Exemplary wound dressings can employ other mesh materials besides polypropylene, including polyethylene and fluorinated materials, such as polyvinylidene fluoride (PVDF). The cover layer may be configured to form a seal around the wound, wherein the cover layer may be moisture vapor permeable. In some embodiments, the wound dressing is vacuum packed. In some embodiments, the wound dressing may include a surfactant.

Exemplary wound dressings can include an activating layer including an acidic hydrogel. Exemplary wound dressings may include an activating layer comprising a composite further comprising a gelling fiber substrate loaded with an acidic hydrogel. Exemplary wound dressings can include substrates such as a DURAFIBER or OPSITE POST-OP pad material. Exemplary wound dressings may contain aqueous Sodium 2-Acrylamido-2-methyl-1-propanesulfonate solution and 2-Acrylamido-2-methyl-1-propanesulfonic acid (SA). Exemplary wound dressings can include a hydrogel with an acidic content of any number within the range of 2.0 SA to 0.5 SA. A 1.0 SA gel can be made of the following components (parts by weight of a 100 g batch): 63.32 g sodium AMPS; 0.0323 g piperazine diacrylamide; 5.436 g AMPS (H+); 31.19 g glycerol and 0.01629 g 2-hydroxy-2-methylpropiophenone. A 0.7 SA gel can be made of the following components (parts by weight of a 100 g batch): 65.28 g sodium AMPS; 0.0317 g piperazine diacrylamide; 3.923 g AMPS (H+); 30.74 g glycerol and 0.01756 g 2-hydroxy-2-methylpropiophenone. Exemplary wound dressings can include an activator layer configured to donate a proton to the nitrite providing layer to produce nitric oxide. Exemplary wound dressings including the nitrite providing layer comprising colocalized sodium nitrite and sodium iso-ascorbate may be configured to produce a ratio of nitric oxide to nitrogen dioxide greater than a wound dressing comprising a nitrite providing layer wherein sodium nitrite and sodium iso-ascorbate are not colocalized. Exemplary wound dressings can include activation of nitric oxide chemistry by fluid in the wound exudate, water provided by the aqueous hydrogel, and/or a separate ampoule of aqueous reagent. Exemplary wound dressings may include methods of application wherein the anhydrous suspension can be loaded into an area of the dressing so as to come into contact with the acidic gel and initiate nitric oxide generation. Exemplary wound dressings may include methods of application wherein the anhydrous suspension and acidic gel are kept separate from each other by a removable handle, and following removal of the handle, the dressing may be manipulated to contact the anhydrous suspension and acidic gel, initiating nitric oxide generation. Exemplary wound dressings may include a pocket comprising a composite layer loaded with an acidic hydrogel, wherein the anhydrous suspension can be loaded into the pocket in order to initiate nitric oxide generation. The anhydrous suspension may be loaded into the pocket as part of a mesh component, or applied directly to the pocket in the absence of a mesh component.

Exemplary wound dressings can include grinding of the sodium nitrite and sodium iso-ascorbate powders to increase surface area of the solid particles in the solvated dispersion, making them more available for reaction and also inhibiting settling out of the suspension. Exemplary wound dressings can include the use of dispersing agents/surfactants etc.

### Terminology

Any patents and applications and other references noted above, including any that may be listed in accompanying filing papers, are incorporated herein by reference. Aspects of the disclosure can be modified, if necessary, to employ the systems, functions, and concepts of the various references described herein to provide yet further implementations.

Features, materials, characteristics, or groups described in conjunction with a particular aspect, embodiment, or example are to be understood to be applicable to any other aspect, embodiment or example described herein unless incompatible therewith. All of the features disclosed in this specification (including any accompanying claims, abstract and drawings), or all of the steps of any method or process so disclosed, may be combined in any combination, except combinations where at least some of such features or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

While certain embodiments have been described, these embodiments have been presented by way of example only, and are not intended to limit the scope of protection. Indeed, the novel methods and systems described herein may be embodied in a variety of other forms. Furthermore, various omissions, substitutions and changes in the form of the methods and systems described herein may be made. Those skilled in the art will appreciate that in some embodiments, the actual steps taken in the processes illustrated or disclosed may differ from those shown in the figures. Depending on the embodiment, certain of the steps described above may be removed, others may be added. For example, the actual steps or order of steps taken in the disclosed processes may differ from those shown in the figure. Depending on the embodiment, certain of the steps described above may be removed, others may be added. Furthermore, the features and attributes of the specific embodiments disclosed above may be combined in different ways to form additional embodiments, all of which fall within the scope of the present disclosure.

Although the present disclosure includes certain embodiments, examples and applications, it will be understood by those skilled in the art that the present disclosure extends beyond the specifically disclosed embodiments to other alternative embodiments or uses and obvious modifications and equivalents thereof, including embodiments which do not provide all of the features and advantages set forth herein. Accordingly, the scope of the present disclosure is not intended to be limited by the described embodiments, and may be defined by claims as presented herein or as presented in the future.

Conditional language, such as “can,” “could,” “might,” or “may,” unless specifically stated otherwise, or otherwise understood within the context as used, is generally intended to convey that certain embodiments include, while other embodiments do not include, certain features, elements, or steps. Thus, such conditional language is not generally intended to imply that features, elements, or steps are in any way required for one or more embodiments or that one or more embodiments necessarily include logic for deciding, with or without user input or prompting, whether these features, elements, or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Likewise, the term “and/or” in reference to a list of two or more items, covers all of the following interpretations of the word: any one of the items in the list, all of the items in the list, and any combination of the items in the list. Further, the term “each,” as used herein, in addition to having its ordinary meaning, can mean any subset of a set of elements to which the term “each” is applied. Additionally, the words “herein,” “above,” “below,” and words of similar import, when used in this application, refer to this application as a whole and not to any particular portions of this application.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

Any of the embodiments described herein can be used with a canister or without a canister. Any of the dressing embodiments described herein can absorb and store wound exudate.

The scope of the present disclosure is not intended to be limited by the description of certain embodiments and may be defined by the claims. The language of the claims is to be interpreted broadly based on the language employed in the claims and not limited to the examples described in the present specification or during the prosecution of the application, which examples are to be construed as non-exclusive.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the disclosure are encompassed in the claim set listed below or presented in the future.

Certain embodiments of the disclosure are encompassed in the claims presented at the end of this specification, or in other claims presented at a later date.

1. A wound treatment apparatus for treating a wound, comprising:
a wound dressing, comprising:
a nitrite providing layer comprising a mesh component, wherein the mesh component comprises a colocalized nitrite salt and an antioxidant/reducing agent,
an activator layer comprising a hydrogel;
wherein the activator layer is configured to donate a proton to the nitrite providing layer to produce nitric oxide.

2. The wound treatment apparatus of claim 1, wherein the nitrite salt and antioxidant/reducing agent are embedded within the mesh component.

3. The wound treatment apparatus of claim 2, wherein the nitrite salt and antioxidant/reducing agent are solubilized, and/or suspended in an anhydrous carrier prior to embedding within the mesh component, wherein the anhydrous carrier is soluble or miscible in water.

4. The wound treatment apparatus of claim 3, wherein the anhydrous water-miscible carrier is selected from the group consisting of polyethylene glycol (PEG) 200, PEG400, PEG600, blends of PEG with different molecular weights, glycerol, triacetin, acetaldehyde, acetic acid, acetone, acetonitrile, 1,2-Butanediol, 1,3-Butanediol, 2-Butoxyethanol, butyric acid, diethanolamine, diethylenetriamine, dimethylformamide, dimethoxyethane, dimethyl sulfoxide, 1,4-Dioxane, ethanol, ethylamine, ethylene glycol, formic acid, furfuryl alcohol, methanol, methyl diethanolamine, methyl isocyanide, N-Methyl-2-pyrrolidone, 1-Propanediol, 1,3-Propanediol, 1,5-Pentanediol, 1-Propanol, 2-Propanol, propanoic acid, propylene glycol, pyridine, tetrahydrofuran, triethylene glycol.

5. The wound treatment apparatus of claim 1, wherein the mesh component of the nitrite providing layer is configured to be stored in a dry state.

6. The wound treatment apparatus of claim 1, wherein the antioxidant/reducing agent colocalized with the nitrite salt in the mesh component of the nitrite providing layer has greater stability than an antioxidant/reducing agent not stored in a dry state.

7. The wound treatment apparatus of claim 1, wherein the antioxidant/reducing agent demonstrates greater stability following sterilization compared to the stability of an antioxidant/reducing agent within a sterilized wound treatment apparatus wherein the antioxidant/reducing agent is not stored in a dry state.

8. The wound treatment apparatus of claim 1, wherein the wound treatment apparatus is configured to develop less coloration than a wound treatment apparatus wherein the antioxidant/reducing agent is not stored in a dry state.

9. The wound treatment apparatus of claim 1, wherein the mesh comprises a coated polypropylene mesh.

10. The wound treatment apparatus of claim 1, wherein the activator layer comprises a composite comprising a gelling fiber substrate loaded with an acidic hydrogel.

11. The wound treatment apparatus of claim 10, wherein the hydrogel further comprises aqueous Sodium 2-Acrylamido-2-methyl-1-propanesulfonate solution and 2-Acrylamido-2-methyl-1-propanesulfonic acid.

12. The wound treatment apparatus of claim 1, wherein the wound treatment apparatus further comprises an acquisition distribution layer.

13. The wound treatment apparatus of claim 1, wherein the wound treatment apparatus further comprises a cover layer configured to form a seal around the wound.

14. The wound treatment apparatus of claim 14, wherein the cover layer is moisture vapor permeable.

15. The wound treatment apparatus of claim 1, wherein the wound treatment apparatus further comprises a masking layer, the masking layer configured to at least partially limit visualization of the wound.

16. The wound treatment apparatus of claim 1, wherein the nitrite salt is sodium nitrite.

17. The wound treatment apparatus of claim 1, wherein the antioxidant/reducing agent comprises sodium iso-ascorbate, iron sulfate, or a derivative of vitamin C.

18. The wound treatment apparatus of claim 1, wherein the wound treatment apparatus further comprises a surfactant.

19. (canceled)

20. A method for treating a wound comprising:
applying a wound dressing to the wound, the wound dressing comprising:
a nitrite providing layer comprising a mesh component, wherein the mesh component comprises a colocalized nitrite salt and antioxidant/reducing agent; and
an activator layer comprising a hydrogel, the activator layer configured to donate a proton to the nitrite providing layer to produce nitric oxide.

21. The method of claim 20, wherein the nitrite salt and antioxidant/reducing agent are embedded within the mesh component.

22-38. (canceled)
